SEQUENCE LISTING (40 sequences)

SEQ ID NO: 1 - DNA, 1176 bp, Saccharomyces cerevisiae
  CDS (1)..(1173): coding for G3PDH
atgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgttttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccaccgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattag

SEQ ID NO: 2 - protein, 391 aa, Saccharomyces cerevisiae
MSAAADRLNLTSGHLNAGRKRSSSSVSLKAAEKPFKVTVIGSGNWGTTIAKVVAENCKGYPEVFAPIVQMWVFEEEINGEKLTEIINTRHQNVKYLPGITLPDNLVANPDLIDSVKDVDIIVFNIPHQFLPRICSQLKGHVDSHVRAISCLKGFEVGAKGVQLLSSYITEELGIQCGALSGANIATEVAQEHWSETTVAYHIPKDFRGEGKDVDHKVLKALFHRPYFHVSVIEDVAGISICGALKNVVALGCGFVEGLGWGNNASAAIQRVGLGEIIRFGQMFFPESREETYYQESAGVADLITTCAGGRNVKVARLMATSGKDAWECEKELLNGQSAQGLITCKEVHEWLETCGSVEDFPLFEAVYQIVYNNYPMKNLPDMIEELDLHED

SEQ ID NO: 3 - DNA, 1299 bp, Saccharomyces cerevisiae
  CDS (1)..(1296): coding for G3PDH
atgcttgctgtcagaagattaacaagatacacattccttaagcgaacgcatccggtgttatatactcgtcgtgcatataaaattttgccttcaagatctactttcctaagaagatcattattacaaacacaactgcactcaaagatgactgctcatactaatatcaaacagcacaaacactgtcatgaggaccatcctatcagaagatcggactctgccgtgtcaattgtacatttgaaacgtgcgcccttcaaggttacagtgattggttctggtaactgggggaccaccatcgccaaagtcattgcggaaaacacagaattgcattcccatatcttcgagccagaggtgagaatgtgggtttttgatgaaaagatcggcgacgaaaatctgacggatatcataaatacaagacaccagaacgttaaatatctacccaatattgacctgccccataatctagtggccgatcctgatcttttacactccatcaagggtgctgacatccttgttttcaacatccctcatcaatttttaccaaacatagtcaaacaattgcaaggccacgtggcccctcatgtaagggccatctcgtgtctaaaagggttcgagttgggctccaagggtgtgcaattgctatcctcctatgttactgatgagttaggaatccaatgtggcgcactatctggtgcaaacttggcaccggaagtggccaaggagcattggtccgaaaccaccgtggcttaccaactaccaaaggattatcaaggtgatggcaaggatgtagatcataagattttgaaattgctgttccacagaccttacttccacgtcaatgtcatcgatgatgttgctggtatatccattgccggtgccttgaagaacgtcgtggcacttgcatgtggtttcgtagaaggtatgggatggggtaacaatgcctccgcagccattcaaaggctgggtttaggtgaaattatcaagttcggtagaatgtttttcccagaatccaaagtcgagacctactatcaagaatccgctggtgttgcagatctgatcaccacctgctcaggcggtagaaacgtcaaggttgccacatacatggccaagaccggtaagtcagccttggaagcagaaaaggaattgcttaacggtcaatccgcccaagggataatcacatgcagagaagttcacgagtggctacaaacatgtgagttgacccaagaattcccaattattcgaggcagtctaccagatagtctacaacaacgtccgcatggaagacctaccggagatgattga

SEQ ID NO: 4 - protein, 432 aa, Saccharomyces cerevisiae
MLAVRRLTRYTFLKRTHPVLYTRRAYKILPSRSTFLRRSLLQTQLHSKMTAHTNIKQHKHCHEDHPIRRSDSAVSIVHLKRAPFKVTVIGSGNWGTTIAKVIAENTELHSHIFEPEVRMWVFDEKIGDENLTDIINTRHQNVKYLPNIDLPHNLVADPDLLHSIKGADILVFNIPHQFLPNIVKQLQGHVAPHVRAISCLKGFELGSKGVQLLSSYVTDELGIQCGALSGANLAPEVAKEHWSETTVAYQLPKDYQGDGKDVDHKILKLLFHRPYFHVNVIDDVAGISIAGALKNVVALACGFVEGMGWGNNASAAIQRLGLGEIIKFGRMFFPESKVETYYQESAGVADLITTCSGGRNVKVATYMAKTGKSALEAEKELLNGQSAQGIITCREVHEWLQTCELTQEFPIIRGSLPDSLQQRPHGRPTGDD

SEQ ID NO: 5 - protein, 384 aa, Saccharomyces cerevisiae
MTAHTNIKQHKHCHEDHPIRRSDSAVSIVHLKRAPFKVTVIGSGNWGTTIAKVIAENTELHSHIFEPEVRMWVFDEKIGDENLTDIINTRHQNVKYLPNIDLPHNLVADPDLLHSIKGADILVFNIPHQFLPNIVKQLQGHVAPHVRAISCLKGFELGSKGVQLLSSYVTDELGIQCGALSGANLAPEVAKEHWSETTVAYQLPKDYQGDGKDVDHKILKLLFHRPYFHVNVIDDVAGISIAGALKNVVALACGFVEGMGWGNNASAAIQRLGLGEIIKFGRMFFPESKVETYYQESAGVADLITTCSGGRNVKVATYMAKTGKSALEAEKELLNGQSAQGIITCREVHEWLQTCELTQEFPIIRGSLPDSLQQRPHGRPTGDD

SEQ ID NO: 6 - DNA, 1122 bp, Schizosaccharomyces pombe
  CDS (1)..(1119): coding for G3PDH
atgactgtggctgctttgaacaaactcagcgctctctccggaagtattcaaaaatctttttcacctaaacttatttctgttggtatcatcggatcaggaaattggggaaccgctattgctaaaatatgtggtgaaaatgccaaggctcatcctgatattttccatcctcaagtacacatgtggatgtatgaagagaagattcaacatgagggaaaagagtgcaatctcacggaagtttttaacactactcatgaaaacgttaaatatctcaaaggtatcaaatgcccttctaacgtcttcgcaaacccggacattcgtgatgtaggttcacgtagcgacattctggtatgggttctccctcaccagttcgttgtgcgtatttgcaatcaattgaagggatgcctaaagaaggatgctgttgcaatttcatgcatcaaaggtgtatctgtcaccaaggaccgtgttcgcctcttttctgatattatcgaagaaaacacgggaatgtattgtggcgttctctctggcgccaacattgccagcgaagttgctcaagagaagttttgcgaaactactatcggatatttgcctaatagttctgttaatccccgctatactcctaagactatccaagctttgtttaaccgtccctacttccgtgtcaacattgttgaggatgttcctggtgttgctttgggcggtgcactcaagaatatcgtcgctgtcgctgccggtattattgatggacttgaattgggagataataccaaatctgctgttatgcgcattggccttctggaaatgcagaaattcggcaggatgtttttcgattgtaagcctcttactatgagcgaggaatcttgtggcatagccgatttaattacaacttgcttaggcggccgtaaccacaaatgcgctgtggcatttgtcaagacaggaaagcccatgcatgttgttgaacaagaacttcttgatggtcagaagttgcaaggtgcagctaccgcgaaggaggtttatgagttccttgataaccagaataaggtaagcgaattcccattgtttacagctgtttatcgcattgtttatgagggacttccacctaataagcttctggaggctatttaa

SEQ ID NO: 7 - protein, 373 aa, Schizosaccharomyces pombe
MTVAALNKLSALSGSIQKSFSPKLISVGIIGSGNWGTAIAKICGENAKAHPDIFHPQVHMWMYEEKIQHEGKECNLTEVFNTTHENVKYLKGIKCPSNVFANPDIRDVGSRSDILVWVLPHQFVVRICNQLKGCLKKDAVAISCIKGVSVTKDRVRLFSDIIEENTGMYCGVLSGANIASEVAQEKFCETTIGYLPNSSVNPRYTPKTIQALFNRPYFRVNIVEDVPGVALGGALKNIVAVAAGIIDGLELGDNTKSAVMRIGLLEMQKFGRMFFDCKPLTMSEESCGIADLITTCLGGRNHKCAVAFVKTGKPMHVVEQELLDGQKLQGAATAKEVYEFLDNQNKVSEFPLFTAVYRIVYEGLPPNKLLEAI

SEQ ID NO: 8 - DNA, 1155 bp, Schizosaccharomyces pombe
  CDS (1)..(1152): coding for G3PDH
atgtctggatatggtcaacaaggtgtttctgctgccaacatcgacagcatccgccccaagaaacgtttgtcaattggtgtagttggctccggtaactggggtactgccattgccaagatttgcggtgaaaatgcccgtgcccacggtcaccatttcagaggtaaggggcgcatgtgggtctttgaggaggagattgagtacaagggtgagaagagaaagctcaccgaagtattcaacgaagctcacgagaatgtcaaatacttacccggcatcgaatgccctcccaacgttattgccgtccccgatgttcgtgaggtcgctagacgtgccgacatccttgtctttgtcgttcctcatcaatttattgaacgcgtttggcaccaaatggtcggtctcattcgccctggtgccgttggtatttcctgtatcaagggtgttgctgtcagcaaggaaggctcgctttactctgaggttatcagcgagaaactcggtatttactgtggtgttctttctggtgctaacgttgcaaacgaagttgcccgtgagcaattctgtgagactactattggtttcaaccctcctaatgaagttgatatccctcgcgagcaaatcgccgccgtctctgatcgcccttacttctcagttgtctccgttgacgacgttgccggtgtcgccttgggtggtgctttgaagaacgtagttgccatggccgttggtttcgctgatggtttggaatggggcggtaataccaaggccgctattatgcgtcgtggtttgttggagatgcaaaagtttgctactaccttcttcgactctgatcctcgtaccatggttgagcaatcttgcggtatcgctgacttggtcacttcttgtttgggtggccgtaacaatcgttgtgctgaagcatttgtcaagactggtaaatctttagagacgcttgaaaaagagctcttaggtggtcaacttcttcaaggagctgccacttccaaggatgttcatgaattccttctcaccaaggatatggtcaaggatttccccttgttcactgccgtttataacatttcctatgaagacatggatcccaaggatttgatcatcgtccttcaaccccttaaggaggactctgagaacgagggcggtactgaaaccgagtaa

SEQ ID NO: 9 - protein, 384 aa, Schizosaccharomyces pombe
MSGYGQQGVSAANIDSIRPKKRLSIGVVGSGNWGTAIAKICGENARAHGHHFRGKGRMWVFEEEIEYKGEKRKLTEVFNEAHENVKYLPGIECPPNVIAVPDVREVARRADILVFVVPHQFIERVWHQMVGLIRPGAVGISCIKGVAVSKEGSLYSEVISEKLGIYCGVLSGANVANEVAREQFCETTIGFNPPNEVDIPREQIAAVSDRPYFSVVSVDDVAGVALGGALKNVVAMAVGFADGLEWGGNTKAAIMRRGLLEMQKFATTFFDSDPRTMVEQSCGIADLVTSCLGGRNNRCAEAFVKTGKSLETLEKELLGGQLLQGAATSKDVHEFLLTKDMVKDFPLFTAVYNISYEDMDPKDLIIVLQPLKEDSENEGGTETE

SEQ ID NO: 10 - DNA, 1197 bp, Yarrowia lipolytica
  CDS (1)..(1194): coding for G3PDH
atgagcgctctacttagatcgtccctgcgttttaaacacatgtccgccgtcaaccgtctcacacaacagcttcgactgctgaccgcctccgcgcctctcagcgcagccaacaccgccggcaaggctcctttcaaggtcgccgttgttggttctggtaactggggaaccaccgtcgccaagattgtcgccgagaactgcactgctcaccccgagctctttgagcccgaggttcgagtctgggttcgagaagagaaggtcaacggcaagaacctgaccgacattttcaacgctgagcacgagaacgtgcgatacctccctaaaatcaaacttcctcacaacctgatcgccgagccggatctgctcaaggccgtcgagggtgccaacatcatcgtcttcaacctgccccatcagttcctggctggtgtctgcaagcagctcaagggccacgtcaaccccaaggctagagccatctcctgcctcaagggtctagatgtcaccccccagggtgtttacctgctctccgacgttatcgagaacgagaccggtctccactgcggtgttctgtccggggctaacctcgccaccgagatcgctctggagaagtactccgagactaccgttgcttacaaccgacccaaggacttctttggcgagggtgatgtgaccaacgatgtgctcaaggctctgttccaccgaccctacttccatgtgcgatgcgttcaggacgtcgccggtgtctccatcggaggtgcccttaagaacgttgttgccctttgcgccggtttcgtcgagggcaagaactggggagacaacgccaaggccgcaattatgcgacgaggcatgcttgagatgatcaacttctccaagcgattcttccccgaaactgatattaacactcttacagtcgagtctgccggtgtggccgatctcatcacctcgtgcgctggaggccgaaacttcaaggtcggccgagcattcggaaaggagagcggctccggcaagaccatccaggacgtggagaaggagcttctcaacggccagtccgcccagggcgtcatcacatgtaacgaggtccacgagctgctcaagaacaagaacatgcagaaggacttccctctgttcgagtccacctggggcattatccacggtgagctcaagattgatgatctccccgagattctttaccacgccaactag

SEQ ID NO: 11 - protein, 398 aa, Yarrowia lipolytica
MSALLRSSLRFKHMSAVNRLTQQLRLLTASAPLSAANTAGKAPFKVAVVGSGNWGTTVAKIVAENCTAHPELFEPEVRVWVREEKVNGKNLTDIFNAEHENVRYLPKIKLPHNLIAEPDLLKAVEGANIIVFNLPHQFLAGVCKQLKGHVNPKARAISCLKGLDVTPQGVYLLSDVIENETGLHCGVLSGANLATEIALEKYSETTVAYNRPKDFFGEGDVTNDVLKALFHRPYFHVRCVQDVAGVSIGGALKNVVALCAGFVEGKNWGDNAKAAIMRRGMLEMINFSKRFFPETDINTLTVESAGVADLITSCAGGRNFKVGRAFGKESGSGKTIQDVEKELLNGQSAQGVITCNEVHELLKNKNMQKDFPLFESTWGIIHGELKIDDLPEILYHAN

SEQ ID NO: 12 - protein, 385 aa, Yarrowia lipolytica
MSAVNRLTQQLRLLTASAPLSAANTAGKAPFKVAVVGSGNWGTTVAKIVAENCTAHPELFEPEVRVWVREEKVNGKNLTDIFNAEHENVRYLPKIKLPHNLIAEPDLLKAVEGANIIVFNLPHQFLAGVCKQLKGHVNPKARAISCLKGLDVTPQGVYLLSDVIENETGLHCGVLSGANLATEIALEKYSETTVAYNRPKDFFGEGDVTNDVLKALFHRPYFHVRCVQDVAGVSIGGALKNVVALCAGFVEGKNWGDNAKAAIMRRGMLEMINFSKRFFPETDINTLTVESAGVADLITSCAGGRNFKVGRAFGKESGSGKTIQDVEKELLNGQSAQGVITCNEVHELLKNKNMQKDFPLFESTWGIIHGELKIDDLPEILYHAN

SEQ ID NO: 13 - DNA, 1206 bp, Zygosaccharomyces rouxii
  CDS (1)..(1203): coding for G3PDH
atggccgctactgacagattaaaccaaacctctgatatcctatcgcaatctatgaagaagaccgactcatcaatgtcagtcgttaccgctgagaatccatacaaagtttccgtcgtcggctctggtaactggggtaccaccatcgccaaggtcgttgccgaaaacaccaaggaaaagccagaattgttccaagaacgtgtggacatgtgggtgtttgaagaacagatcgacggtactccattggcccaaatcatcaacaccaagcaccagaacgtgaaatacttgccaaacatcgaccttccggacaatttggtcgctaacccagacttgattgccaccacgaaggacgccgatgtgattgttttcaacgttccccatcaatttttgggccgtatcgttgctcaaatgaagggtcaaatcaaaccaactgcacgtgcggtctcctgtctaaagggtttcgaagttggtccaaagggtgtgcagcttctatctgactacgtcactcaagaattgggtatcgaatgtggtgctctatctggtgctaacttggccccagaagtcgccaaggaacactggtccgagaccaccgtcgcttaccacatcccagacgacttcaagggtgacggtaaggacatcgaccaccgtgtcttgaagcagttgttccacagaccatacttccacgtgaatgtgattgacgatgttgctggtatctccatcgcaggtgcattgaagaacgtggtcgccttgggttgcggtttcgttaccggtctaggttggggtaacaacgccgccgccgccatccaacgtgtcggtttgggtgaaatcatcaagttcggtaggatgttcttcccagaatccaaggtggagacttactaccaagaatccgcaggtgttgctgacttgatcaccacctgttccggtggtagaaacgtccgtgttgccaccgaaatggccaagactggtaagagcggtgagcaagtcgaaaaagacatcttgaacggtcaatccgctcaaggtttggtcacctgtaaggaagttcaccagtggttagaatctagtggaaacaccgaagacttcccattgttcgaggctgtctaccagatcacttacgaaaacgtgcccatgaaggagttgccatctatgatcgaagaattggatatcgatagcacatcgaagtgcgtattgagttacaagatgggtctctag

SEQ ID NO: 14 - protein, 401 aa, Zygosaccharomyces rouxii
MAATDRLNQTSDILSQSMKKTDSSMSVVTAENPYKVSVVGSGNWGTTIAKVVAENTKEKPELFQERVDMWVFEEQIDGTPLAQIINTKHQNVKYLPNIDLPDNLVANPDLIATTKDADVIVFNVPHQFLGRIVAQMKGQIKPTARAVSCLKGFEVGPKGVQLLSDYVTQELGIECGALSGANLAPEVAKEHWSETTVAYHIPDDFKGDGKDIDHRVLKQLFHRPYFHVNVIDDVAGISIAGALKNVVALGCGFVTGLGWGNNAAAAIQRVGLGEIIKFGRMFFPESKVETYYQESAGVADLITTCSGGRNVRVATEMAKTGKSGEQVEKDILNGQSAQGLVTCKEVHQWLESSGNTEDFPLFEAVYQITYENVPMKELPSMIEELDIDSTSKCVLSYKMGL

SEQ ID NO: 15 - DNA, 1170 bp, Zygosaccharomyces rouxii
  CDS (1)..(1167): coding for G3PDH
atggccgccactgacagattaaaccaaacctccgatatcctatctcattctatgaagaagactgatacctcaatgtcaattgttaccgctgagaatccttacaaggtcgctgttgtcggttctggtaactggggtaccactatcgctaaggttgttgccgaaaacaccaaagaaaagccagagttgttccaaggacgtgtggacatgtgggttttcgaagaacaaatcgatggtactccattgactcaaatcatcaacaccaaacaccaaaacgtcaaataccttccaaacatcgatcttccggggaatttggtcgctaacccagatttgatctctactaccaaggacgctgatgtcatcgttttcaacgttcctcaccaatttttgggccgtatcgtttctcaaatgaagggtcaaatcaaaccagatgctcgtgccatctcctgtctaaagggtttcgaagttggtccaaagggtgtccaactactttctgactacgtcactcaagaattaggtatccaatgtggtgccctatctggtgctaacttggctccagaagtcgccaaggaacactggtccgaaactaccgtcgcttaccaagtcccagatgacttcaagggtgaaggtaaagatatcgaccaccgtgtcttgaaacaattgttccacagaccatacttccacgtcaatgtgatcgacgatgttgctggtatttctatcgcaggtgcattgaagaacgtggttgccttgggttgcggtttcgtcaccggtctaggctggggtaacaacgctgccgccgccatccaacgtgttggtttgggtgaaatcatcaagttcggtagaatgttcttcccagaatccaaggtggaaacttactaccaagaatctgcaggtgttgctgatttgatcactacctgttccggtggtagaaacgttcgtgtcgccactgaaatggccaagactggtaagagcggtgaacaagtcgaaaaggacatcttgaacggtcaatccgctcaaggtttgattactgctaaggaagtccaccaatggttggaatccagcggtcacaccgaagaatacccattgtttgaagccgtctaccaaatcacttacgaaaacgtgcccatgaaggagttgccatccatgatcgaagaattggatatcgtagaataa

SEQ ID NO: 16 - protein, 389 aa, Zygosaccharomyces rouxii
MAATDRLNQTSDILSHSMKKTDTSMSIVTAENPYKVAVVGSGNWGTTIAKVVAENTKEKPELFQGRVDMWVFEEQIDGTPLTQIINTKHQNVKYLPNIDLPGNLVANPDLISTTKDADVIVFNVPHQFLGRIVSQMKGQIKPDARAISCLKGFEVGPKGVQLLSDYVTQELGIQCGALSGANLAPEVAKEHWSETTVAYQVPDDFKGEGKDIDHRVLKQLFHRPYFHVNVIDDVAGISIAGALKNVVALGCGFVTGLGWGNNAAAAIQRVGLGEIIKFGRMFFPESKVETYYQESAGVADLITTCSGGRNVRVATEMAKTGKSGEQVEKDILNGQSAQGLITAKEVHQWLESSGHTEEYPLFEAVYQITYENVPMKELPSMIEELDIVE

SEQ ID NO: 17 - DNA, 8809 bp, Artificial sequence
  Description of artificial sequence expression vector pSUN-USP containing Saccharomyces G3PDH
aatattcaaacaaacacatacagcgcgacttatcatggacatacaaatggacgaacggataaaccttttcacgcccttttaaatatccgattattctaataaacgctcttttctcttaggtttacccgccaatatatcctgtcaaacactgatagtttaaactgaaggcgggaaacgacaatcagatctagtaggaaacagctatgaccatgattacgccaagcttgcatgcctgcaggtcgactctagactagtggatccgatatcgcccgggctcgaggtaccgagctcgaattcggcgcgccgagctcctcgagcaaatttacacattgccactaaacgtctaaacccttgtaatttgtttttgttttactatgtgtgttatgtatttgatttgcgataaatttttatatttggtactaaatttataacaccttttatgctaacgtttgccaacacttagcaatttgcaagttgattaattgattctaaattatttttgtcttctaaatacatatactaatcaactggaaatgtaaatatttgctaatatttctactataggagaattaaagtgagtgaatatggtaccacaaggtttggagatttaattgttgcaatgatgcatggatggcatatacaccaaacattcaataattcttgaggataataatggtaccacacaagatttgaggtgcatgaacgtcacgtggacaaaaggtttagtaatttttcaagacaacaatgttaccacacacaagttttgaggtgcatgcatggatgccctgtggaaagtttaaaaatattttggaaatgatttgcatggaagccatgtgtaaaaccatgacatccacttggaggatgcaataatgaagaaaactacaaatttacatgcaactagttatgcatgtagtctatataatgaggattttgcaatactttcattcatacacactcactaagttttacacgattataatttcttcatagccagcccaccgcggtgggcggccgccatgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgtcttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccactgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattaggcggccgcctgcagtctagaaggcctcctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtccgtcgacgaattcactggccgtcgttttacaacgactcagagcttgacaggaggcccgatctagtaacatagatgacaccgcgcgcgataatttatcctagtttgcgcgctatattttgttttctatcgcgtattaaatgtataattgcgggactctaatcataaaaacccatctcataaataacgtcatgcattacatgttaattattacatgcttaacgtaattcaacagaaattatatgataatcatcgcaagaccggcaacaggattcaatcttaagaaactttattgccaaatgtttgaacgatcggggatcatccgggtctgtggcgggaactccacgaaaatatccgaacgcagcaagatctagagcttgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgtgtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatccagatccggtgcagattatttggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcagattgtcgtttcccgccttcagtttaaactatcagtgtttgacaggatcactgcttggtaataattgtcattagattgtttttatgcatagatgcactcgaaatcagccaattttagacaagtatcaaacggatgttaattcagtacattaaagacgtccgcaatgtgttattaagttgtctaagcgtcaatttgtttacaccacaatatatcctgccaccagccagccaacagctccccgaccggcagctcggcacaaaatcaccacgcgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgagcggtcgcaaaccatccggcccggtacaaatcggcgcggcgctgggtgatgacctggtggagaagttgaaggccgcgcaggccgcccagcggcaacgcatcgaggcagaagacgccccggtgaatcgtggcaaggggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggtctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagccgaagagctgcaaaaagcgcctacccttcggtcgctgcgctccctacgccccgccgcttcgcgtcggcctatcgcggcctatgcggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcatgatatatctcccaatttgtgtagggcttattatgcacgcttaaaaataataaaagcagacttgacctgatagtttggctgtgagcaattatgtgcttagtgcatctaacgcttgagttaagccgcgccgcgaagcggcgtcggcttgaacgaatttctagctagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttcccccatgatgtttaactttgttttagggcgactgccctgctgcgtaacatcgttgctgctccataacatcaaacatcgacccacggcgtaacgcgcttgctgcttggatgcccgaggcatagactgtaccccaaaaaaacagtcataacaagccatgaaaaccgccactgcgttccatg

SEQ ID NO: 18 - DNA, 26 bp, Artificial sequence
  Description of artificial sequence oligonucleotide primer
actagtatgtctgctgctgctgatag

SEQ ID NO: 19 - DNA, 26 bp, Artificial sequence
  Description of artificial sequence oligonucleotide primer
ctcgagatcttcatgtagatctaatt

SEQ ID NO: 20 - DNA, 29 bp, Artificial sequence
  Description of artificial sequence oligonucleotide primer
gcggccgccatgtctgctgctgctgatag

SEQ ID NO: 21 - DNA, 28 bp, Artificial sequence
  Description of artificial sequence oligonucleotide primer
gcggccgcatcttcatgtagatctaatt

SEQ ID NO: 22 - protein, 11 aa, Artificial sequence
  Description of artificial sequence Yeast G3PDH sequence motive
GSGNWGTXIAK

SEQ ID NO: 23 - protein, 8 aa, Artificial sequence
  Description of artificial sequence Yeast G3PDH sequence motive
HXQNVKYL

SEQ ID NO: 24 - protein, 12 aa, Artificial sequence
  Description of artificial sequence Yeast G3PDH sequence motive
XXXVXXXPHQFX

SEQ ID NO: 25 - protein, 7 aa, Artificial sequence
  Description of artificial sequence Yeast G3PDH sequence motive
XXSCXKG

SEQ ID NO: 26 - protein, 14 aa, Artificial sequence
  Description of artificial sequence Yeast G3PDH sequence motive
CGXLSGANXAXEXA

SEQ ID NO: 27 - protein, 9 aa, Artificial sequence
  Description of artificial sequence Yeast G3PDH sequence motive
XFXRPYFXV

SEQ ID NO: 28 - protein, 9 aa, Artificial sequence
  Description of artificial sequence Yeast G3PDH sequence motive
GXXEXXXFX

SEQ ID NO: 29 - protein, 16 aa, Artificial sequence
  Description of artificial sequence Yeast G3PDH sequence motive
GSGNWGTTIAKVXAEN

SEQ ID NO: 30 - protein, 11 aa, Artificial sequence
  Description of artificial sequence Yeast G3PDH sequence motive
NTXHQNVKYLP

SEQ ID NO: 31 - protein, 12 aa, Artificial sequence
  Description of artificial sequence Yeast G3PDH sequence motive
DXLVFNXPHQFL

SEQ ID NO: 32 - protein, 10 aa, Artificial sequence
  Description of artificial sequence Yeast G3PDH sequence motive
RAXSCLKGFE

SEQ ID NO: 33 - protein, 14 aa, Artificial sequence
  Description of artificial sequence Yeast G3PDH sequence motive
CGALSGANLAXEVA

SEQ ID NO: 34 - protein, 9 aa, Artificial sequence
  Description of artificial sequence Yeast G3PDH sequence motive
LFHRPYFHV

SEQ ID NO: 35 - protein, 9 aa, Artificial sequence
  Description of artificial sequence Yeast G3PDH sequence motive
GLGEIIXFG

SEQ ID NO: 36 - DNA, 13718 bp, Artificial sequence
  Description of artificial sequence expression vector pGPTV-gpd1
gatctggcgccggccagcgagacgagcaagattggccgccgcccgaaacgatccgacagcgcgcccagcacaggtgcgcaggcaaattgcaccaacgcatacagcgccagcagaatgccatagtgggcggtgacgtcgttcgagtgaaccagatcgcgcaggaggcccggcagcaccggcataatcaggccgatgccgacagcgtcgagcgcgacagtgctcagaattacgatcaggggtatgttgggtttcacgtctggcctccggaccagcctccgctggtccgattgaacgcgcggattctttatcactgataagttggtggacatattatgtttatcagtgataaagtgtcaagcatgacaaagttgcagccgaatacagtgatccgtgccgccctggacctgttgaacgaggtcggcgtagacggtctgacgacacgcaaactggcggaacggttgggggttcagcagccggcgctttactggcacttcaggaacaagcgggcgctgctcgacgcactggccgaagccatgctggcggagaatcatacgcattcggtgccgagagccgacgacgactggcgctcatttctgatcgggaatgcccgcagcttcaggcaggcgctgctcgcctaccgcgatggcgcgcgcatccatgccggcacgcgaccgggcgcaccgcagatggaaacggccgacgcgcagcttcgcttcctctgcgaggcgggtttttcggccggggacgccgtcaatgcgctgatgacaatcagctacttcactgttggggccgtgcttgaggagcaggccggcgacagcgatgccggcgagcgcggcggcaccgttgaacaggctccgctctcgccgctgttgcgggccgcgatagacgccttcgacgaagccggtccggacgcagcgttcgagcagggactcgcggtgattgtcgatggattggcgaaaaggaggctcgttgtcaggaacgttgaaggaccgagaaagggtgacgattgatcaggaccgctgccggagcgcaacccactcactacagcagagccatgtagacaacatcccctccccctttccaccgcgtcagacgcccgtagcagcccgctacgggctttttcatgccctgccctagcgtccaagcctcacggccgcgctcggcctctctggcggccttctggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagctggcccgcatcaatggcgacctgggccgcctgggcggcctgctgaaactctggctcaccgacgacccgcgcacggcgcggttcggtgatgccacgatcctcgccctgctggcgaagatcgaagagaagcaggacgagcttggcaaggtcatgatgggcgtggtccgcccgagggcagagccatgacttttttagccgctaaaacggccggggggtgcgcgtgattgccaagcacgtccccatgcgctccatcaagaagagcgacttcgcggagctggtgaagtacatcaccgacgagcaaggcaagaccgagcgcctttgcgacgctcaccgggctggttgccctcgccgctgggctggcggccgtctatggccctgcaaacgcgccagaaacgccgtcgaagccgtgtgcgagacaccgcggccgccggcgttgtggatacctcgcggaaaacttggccctcactgacagatgaggggcggacgttgacacttgaggggccgactcacccggcgcggcgttgacagatgaggggcaggctcgatttcggccggcgacgtggagctggccagcctcgcaaatcggcgaaaacgcctgattttacgcgagtttcccacagatgatgtggacaagcctggggataagtgccctgcggtattgacacttgaggggcgcgactactgacagatgaggggcgcgatccttgacacttgaggggcagagtgctgacagatgaggggcgcacctattgacatttgaggggctgtccacaggcagaaaatccagcatttgcaagggtttccgcccgtttttcggccaccgctaacctgtcttttaacctgcttttaaaccaatatttataaaccttgtttttaaccagggctgcgccctgtgcgcgtgaccgcgcacgccgaaggggggtgcccccccttctcgaaccctcccggcccgctaacgcgggcctcccatccccccaggggctgcgcccctcggccgcgaacggcctcaccccaaaaatggcagcgctggcagtccttgccattgccgggatcggggcagtaacgggatgggcgatcagcccgagcgcgacgcccggaagcattgacgtgccgcaggtgctggcatcgacattcagcgaccaggtgccgggcagtgagggcggcggcctgggtggcggcctgcccttcacttcggccgtcggggcattcacggacttcatggcggggccggcaatttttaccttgggcattcttggcatagtggtcgcgggtgccgtgctcgtgttcgggggtgcgataaacccagcgaaccatttgaggtgataggtaagattataccgaggtatgaaaacgagaattggacctttacagaattactctatgaagcgccatatttaaaaagctaccaagacgaagaggatgaagaggatgaggaggcagattgccttgaatatattgacaatactgataagataatatatcttttatatagaagatatcgccgtatgtaaggatttcagggggcaaggcataggcagcgcgcttatcaatatatctatagaatgggcaaagcataaaaacttgcatggactaatgcttgaaacccaggacaataaccttatagcttgtaaattctatcataattgggtaatgactccaacttattgatagtgttttatgttcagataatgcccgatgactttgtcatgcagctccaccgattttgagaacgacagcgacttccgtcccagccgtgccaggtgctgcctcagattcaggttatgccgctcaattcgctgcgtatatcgcttgctgattacgtgcagctttcccttcaggcgggattcatacagcggccagccatccgtcatccatatcaccacgtcaaagggtgacagcaggctcataagacgccccagcgtcgccatagtgcgttcaccgaatacgtgcgcaacaaccgtcttccggagactgtcatacgcgtaaaacagccagcgctggcgcgatttagccccgacatagccccactgttcgtccatttccgcgcagacgatgacgtcactgcccggctgtatgcgcgaggttaccgactgcggcctgagttttttaagtgacgtaaaatcgtgttgaggccaacgcccataatgcgggctgttgcccggcatccaacgccattcatggccatatcaatgattttctggtgcgtaccgggttgagaagcggtgtaagtgaactgcagttgccatgttttacggcagtgagagcagagatagcgctgatgtccggcggtgcttttgccgttacgcaccaccccgtcagtagctgaacaggagggacagctgatagacacagaagccactggagcacctcaaaaacaccatcatacactaaatcagtaagttggcagcatcacccataattgtggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagatgtggcgcaacgatgccggcgacaagcaggagcgcaccgacttcttccgcatcaagtgttttggctctcaggccgaggcccacggcaagtatttgggcaaggggtcgctggtattcgtgcagggcaagattcggaataccaagtacgagaaggacggccagacggtctacgggaccgacttcattgccgataaggtggattatctggacaccaaggcaccaggcgggtcaaatcaggaataagggcacattgccccggcgtgagtcggggcaatcccgcaaggagggtgaatgaatcggacgtttgaccggaaggcatacaggcaagaactgatcgacgcggggttttccgccgaggatgccgaaaccatcgcaagccgcaccgtcatgcgtgcgccccgcgaaaccttccagtccgtcggctcgatggtccagcaagctacggccaagatcgagcgcgacagcgtgcaactggctccccctgccctgcccgcgccatcggccgccgtggagcgttcgcgtcgtctcgaacaggaggcggcaggtttggcgaagtcgatgaccatcgacacgcgaggaactatgacgaccaagaagcgaaaaaccgccggcgaggacctggcaaaacaggtcagcgaggccaagcaggccgcgttgctgaaacacacgaagcagcagatcaaggaaatgcagctttccttgttcgatattgcgccgtggccggacacgatgcgagcgatgccaaacgacacggcccgctctgccctgttcaccacgcgcaacaagaaaatcccgcgcgaggcgctgcaaaacaaggtcattttccacgtcaacaaggacgtgaagatcacctacaccggcgtcgagctgcgggccgacgatgacgaactggtgtggcagcaggtgttggagtacgcgaagcgcacccctatcggcgagccgatcaccttcacgttctacgagctttgccaggacctgggctggtcgatcaatggccggtattacacgaaggccgaggaatgcctgtcgcgcctacaggcgacggcgatgggcttcacgtccgaccgcgttgggcacctggaatcggtgtcgctgctgcaccgcttccgcgtcctggaccgtggcaagaaaacgtcccgttgccaggtcctgatcgacgaggaaatcgtcgtgctgtttgctggcgaccactacacgaaattcatatgggagaagtaccgcaagctgtcgccgacggcccgacggatgttcgactatttcagctcgcaccgggagccgtacccgctcaagctggaaaccttccgcctcatgtgcggatcggattccacccgcgtgaagaagtggcgcgagcaggtcggcgaagcctgcgaagagttgcgaggcagcggcctggtggaacacgcctgggtcaatgatgacctggtgcattgcaaacgctagggccttgtggggtcagttccggctgggggttcagcagccagcgctttactggcatttcaggaacaagcgggcactgctcgacgcacttgcttcgctcagtatcgctcgggacgcacggcgcgctctacgaactgccgataaacagaggattaaaattgacaattgtgattaaggctcagattcgacggcttggagcggccgacgtgcaggatttccgcgagatccgattgtcggccctgaagaaagctccagagatgttcgggtccgtttacgagcacgaggagaaaaagcccatggaggcgttcgctgaacggttgcgagatgccgtggcattcggcgcctacatcgacggcgagatcattgggctgtcggtcttcaaacaggaggacggccccaaggacgctcacaaggcgcatctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagccccagatctggggtcgatcagccggggatgcatcaggccgacagtcggaacttcgggtccccgacctgtaccattcggtgagcaatggataggggagttgatatcgtcaacgttcacttctaaagaaatagcgccactcagcttcctcagcggctttatccagcgatttcctattatgtcggcatagttctcaagatcgacagcctgtcacggttaagcgagaaatgaataagaaggctgataattcggatctctgcgagggagatgatatttgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggtttttcttttcaccagtgagacgggcaacagctgattgcccttcaccgcctggccctgagagagttgcagcaagcggtccacgctggtttgccccagcaggcgaaaatcctgtttgatggtggttccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattaattcccatcttgaaagaaatatagtttaaatatttattgataaaataacaagtcaggtattatagtccaagcaaaaacataaatttattgatgcaagtttaaattcagaaatatttcaataactgattatatcagctggtacattgccgtagatgaaagactgagtgcgatattatgtgtaatacataaattgatgatatagctagcttagctcatcgggggatccgtcgaagctagcttgggtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatccaagctcccatgggccctcgactagagtcgagatctggattgagagtgaatatgagactctaattggataccgaggggaatttatggaacgtcagtggagcatttttgacaagaaatatttgctagctgatagtgaccttaggcgacttttgaacgcgcaataatggtttctgacgtatgtgcttagctcattaaactccagaaacccgcggctgagtggctccttcaacgttgcggttctgtcagttccaaacgtaaaacggcttgtcccgcgtcatcggcgggggtcataacgtgactcccttaattctccgctcatgatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctttcttcatcggtgattgattcctttaaagacttatgtttcttatcttgcttctgaggcaagtattcagttaccagttaccacttatattctggactttctgactgcatcctcatttttccaacattttaaatttcactattggctgaatgcttcttctttgaggaagaaacaattcagatggcagaaatgtatcaaccaatgcatatatacaaatgtacctcttgttctcaaaacatctatcggatggttccatttgctttgtcatccaattagtgactactttatattattcactcctctttattactattttcatgcgaggttgccatgtacattatatttgtaaggattgacgctattgagcgtttttcttcaattttctttattttagacatgggtatgaaatgtgtgttagagttgggttgaatgagatatacgttcaagtgaatggcataccgttctcgagtaaggatgacctacccattcttgagacaaatgttacattttagtatcagagtaaaatgtgtacctataactcaaattcgattgacatgtatccattcaacataaaattaaaccagcctgcacctgcatccacatttcaagtattttcaaaccgttcggctcctatccaccgggtgtaacaagacggattccgaatttggaagattttgactcaaattcccaatttatattgaccgtgactaaatcaactttaacttctataattctgattaagctcccaatttatattcccaacggcactacctccaaaatttatagactctcatccccttttaaaccaacttagtaaacgtttttttttttaattttatgaagttaagtttttaccttgtttttaaaaagaatcgttcataagatgccatgccagaacattagctacacgttacacatagcatgcagccgcggagaattgtttttcttcgccacttgtcactcccttcaaacacctaagagcttctctctcacagcacacacatacaatcacatgcgtgcatgcattattacacgtgatcgccatgcaaatctcctttatagcctataaattaactcatccgcttcactctttactcaaaccaaaactcatcaatacaaacaagattaaaaacatacacgaggatccactagtatgtctgctgctgctgatagattaaacttaacttccggccacttgaatgctggtagaaagagaagttcctcttctgtttctttgaaggctgccgaaaagcctttcaaggttactgtgattggatctggtaactggggtactactattgccaaggtggttgccgaaaattgtaagggatacccagaagttttcgctccaatagtacaaatgtgggtgttcgaagaagagatcaatggtgaaaaattgactgaaatcataaatactagacatcaaaacgtgaaatacttgcctggcatcactctacccgacaatttggttgctaatccagacttgattgattcagtcaaggatgtcgacatcatcgtcttcaacattccacatcaatttttgccccgtatctgtagccaattgaaaggtcatgttgattcacacgtcagagctatctcctgtctaaagggttttgaagttggtgctaaaggtgtccaattgctatcctcttacatcactgaggaactaggtattcaatgtggtgctctatctggtgctaacattgccactgaagtcgctcaagaacactggtctgaaacaacagttgcttaccacattccaaaggatttcagaggcgagggcaaggacgtcgaccataaggttctaaaggccttgttccacagaccttacttccacgttagtgtcatcgaagatgttgctggtatctccatctgtggtgctttgaagaacgttgttgccttaggttgtggtttcgtcgaaggtctaggctggggtaacaacgcttctgctgccatccaaagagtcggtttgggtgagatcatcagattcggtcaaatgtttttcccagaatctagagaagaaacatactaccaagagtctgctggtgttgctgatttgatcaccacctgcgctggtggtagaaacgtcaaggttgctaggctaatggctacttctggtaaggacgcctgggaatgtgaaaaggagttgttgaatggccaatccgctcaaggtttaattacctgcaaagaagttcacgaatggttggaaacatgtggctctgtcgaagacttcccattatttgaagccgtataccaaatcgtttacaacaactacccaatgaagaacctgccggacatgattgaagaattagatctacatgaagattagctcgacgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattcagatcggctgagtggctccttcaacgttgcggntctgtcagtnccaaacgtaaaacgggttggtccgcggnatcgggcggggggccttaaccgtgnactnccntnattnctccggcttcantgnnnagaattggncntttccccgncntcagtttaaactatcaggtgtttgacaggatatatttggcgggtaaacctaaganaaaagagcgtttattagaataatcggatatttaaaagggccgngaaaaggtttatcccttccgtccatttgtatgngcatgccnaccaccagggttcccca

SEQ ID NO: 37 - DNA, 1254 bp, Emericella nidulans
  CDS (1)..(1251): coding for G3PDH
atgggctctcttggaccgtataagcaaaagcacaaggtgactgtggtgggatcgggtaactggggcaccgctatagccaaaatcgtcgccgagaatactgccagcaaccctgcggtctttgagaaggatgttcagatgtgggttttcgaggaaaaggtcgagattccgaaatcgtcgaagcattatgatcctgcctcttctctttgccagggcccgcagaatctgacagatattatcaaccatacccatgagaatatcaagtacctccccggaattacccttccggaaaacttgattgccaatccatcgctagtcgacgcggttcaagacagcactatcctcgtcttcaacctaccccatcaattcatcatcaatatttgtgaacagatcaagggcaagattgtcccatacgcgcgtggaatttcttgcataaagggcgtggatgtgaatgaggaaggagtccacctgttttccgaaacaattggaaagattctcgggatctactgtggcgccctgtccggtgccaacatcgcgaatgaggtcgcccaggaaaagtggtccgagtctagcattggttatgatccaccgcattttgactctaaagccccttctcctcccaaccgatccccttccgcatcgactgacaatatcctgcacttcgagcacaaagacgtttcgggtcaactttcgcgggtaaagctacaggctctaccttccgaatttcctcccatcgaccatgcccttctcaagtcgctattccaccgtccttacttccatattggtgtggtaagtgacgtcgcaggtgtttcgttaggaggtgcccttaagaatgtcgttgctgtcgcggcagggtgggttgtgggcaaaggatggggagacaatgcgaaggctgcaattatgcgagttgggcttttggaaatggtgaagttcggcgaacagtttttcggtgctaccatcaacactcgcaccttcactgaagaaagtgctggtgttgccgatctaatcacgagttgcagtggcggacgaaacttccgctgcgcaaagcttagcattgaaagaaaccagccgattgagaaaatcgaggagacagagttgaacggccagaagctgcaaggcactttgactgcagtcgaagtcaacagtttcttgaaaaagcaaggtttagaagaagagttcccattgtttactgcagtctaccgagttcttcaaggcaccatgtctgtggacgagattccttctttcattgagcggtaa

SEQ ID NO: 38 - protein, 417 aa, Emericella nidulans
MGSLGPYKQKHKVTVVGSGNWGTAIAKIVAENTASNPAVFEKDVQMWVFEEKVEIPKSSKHYDPASSLCQGPQNLTDIINHTHENIKYLPGITLPENLIANPSLVDAVQDSTILVFNLPHQFIINICEQIKGKIVPYARGISCIKGVDVNEEGVHLFSETIGKILGIYCGALSGANIANEVAQEKWSESSIGYDPPHFDSKAPSPPNRSPSASTDNILHFEHKDVSGQLSRVKLQALPSEFPPIDHALLKSLFHRPYFHIGVVSDVAGVSLGGALKNVVAVAAGWVVGKGWGDNAKAAIMRVGLLEMVKFGEQFFGATINTRTFTEESAGVADLITSCSGGRNFRCAKLSIERNQPIEKIEETELNGQKLQGTLTAVEVNSFLKKQGLEEEFPLFTAVYRVLQGTMSVDEIPSFIER

SEQ ID NO: 39 - DNA, 999 bp, Debaryomyces hansenii
  CDS (1)..(996): coding for G3PDH (partial)
ggatctggtaactggggtactgctgttgctaagatcgtatctgaaaacacggctgaaaaaccagaagtgttcgaaaagcaagtgaacatgtgggtttttgaagaagaagttgacggacaaaagttgactgaaatcatcaacgccaaacacgaaaacgttaagtacttgccagaagtcaagttgccggaaaacttggttgcaaacccagacgttgttgacactgtcaaggatgcagacttattaatttttaacattccacatcaattcttaccaagagtgtgtaagcaattggttggccatgtcaagccatctgccagagccatctcctgtttgaagggtttggaagttggcccagaaggttgtaagttgttatcgcaatctatcaacgatactttaggtgtccactgtggtgtcttatctggtgccaacattgccaacgaagttgccagagaaagatggtctgaaaccaccattgcctacaacattccagaagatttcagaggtaagggtagagatatcgacgaatacgtcttaaagcaattattccacagaacctacttccatgtcagagtcatcaacgacatcataggtgcttctttcgctggtgctttgaagaatgttgttgcctgtgctgttggtttcgttatcggtgccggctggggtgacaacgctaaggccgctatcatgagaatcggtatcagagaaatcatccactttgcctcttactaccaaaagttcggtgtcaagggtccagctccagaatccactactttcactgaggaatctgccggtgtcgctgacttaatcaccacttgttccggtggtagaaatgtcaaggttgctagatacatgattgaaaacaacgttgacgcttgggaagccgaaaagattgtcttaaagggtcaatcttctcaaggtatcttaactgccaaggaagtccacgaattgttaactaactacaacttatcgaatgaattcccattatttgaagccgtatac

SEQ ID NO: 40 - protein, 332 aa, Debaryomyces hansenii
GSGNWGTAVAKIVSENTAEKPEVFEKQVNMWVFEEEVDGQKLTEIINAKHENVKYLPEVKLPENLVANPDVVDTVKDADLLIFNIPHQFLPRVCKQLVGHVKPSARAISCLKGLEVGPEGCKLLSQSINDTLGVHCGVLSGANIANEVARERWSETTIAYNIPEDFRGKGRDIDEYVLKQLFHRTYFHVRVINDIIGASFAGALKNVVACAVGFVIGAGWGDNAKAAIMRIGIREIIHFASYYQKFGVKGPAPESTTFTEESAGVADLITTCSGGRNVKVARYMIENNVDAWEAEKIVLKGQSSQGILTAKEVHELLTNYNLSNEFPLFEAV

(19) United States
(12) Patent Application Publication
(10) Pub. No.: US 2009/0083882 A1
(43) Pub. Date: Mar. 26, 2009

(54) METHOD FOR INCREASING THE TOTAL OIL CONTENT IN OIL PLANTS

(75) Inventors: Thorsten Zank, Mannheim (DE); Oliver Oswald, Lautertal (DE); Jorg Bauer, Limburgerhof (DE); Helene Vigeolas, Potsdam (DE); Peter Geigenberger, Berlin (DE); Peter Waldeck, Potsdam (DE); Mark Stitt, Potsdam (DE)

Correspondence Address: CONNOLLY BOVE LODGE & HUTZ, LLP, P O BOX 2207, WILMINGTON, DE, 19899, US

(73) Assignees: Max-Planck-Gesellschaft zur Forderung der Wissenschaften e.V., Munchen (DE); BASF Plant Science GmbH, Ludwigshafen (DE)

(21) Appl. No.: 12/092,603

(22) PCT Filed: Nov. 6, 2006
(86) PCT No.: PCT/EP06/10592

§ 371 Date: May 5, 2008

(30) Foreign Application Priority Data
Nov. 7, 2005 (DE) 10 2005 053 318.3

(51) Int. Cl. C12N 15/82; A01H 1/04

(52) U.S. Cl. 800/281

(57) ABSTRACT

The invention relates to methods of increasing the total oil content and/or the glycerol 3-phosphate content in transgenic oil crop plants which comprise at least 20% by weight of oleic acid based on the total fatty acid content, preferably in plant seeds, by expressing glycerol 3-phosphate dehydrogenases (G3PDHs) from yeasts, preferably from Saccharomyces cerevisiae. The oil and/or the free fatty acids obtained in the process are advantageously added to polymers, foodstuffs, feedstuffs, cosmetics, pharmaceuticals or products with industrial applications.

The invention relates to methods of increasing the total oil content and/or the glycerol 3-phosphate content in transgenic oil crop plants which comprise at least 20% by weight of oleic acid based on the total fatty acid content, preferably in plant seeds, by expressing glycerol 3-phosphate dehydrogenases (G3PDHs) from yeasts, preferably from Saccharomyces cerevisiae. The oil and/or the free fatty acids obtained in the process are advantageously added to polymers, foodstuffs, feedstuffs, cosmetics, pharmaceuticals or products with industrial applications.

Increasing the total oil content and/or the glycerol 3-phosphate content in transgenic oil crop plants and in particular in plant seeds is of great interest both for traditional and modern plant breeding and in particular for plant biotechnology. Owing to the increasing consumption of vegetable oils for nutrition or industrial applications, possibilities of increasing or modifying vegetable oils are increasingly the subject of current research [for example Töpfer et al. (1995) Science 268:681-686] Its aim is in particular increasing the fatty acid content in seed oils.

The fatty acids which can be obtained from the vegetable oils are also of particular interest. They are employed, for example, as bases for plasticizers, lubricants, surfactants, cosmetics and the like and are employed as valuable feedstocks in the food and feed industries. Thus, for example, it is of particular interest to provide rapeseed oils with fatty acids with medium chain length since these are in demand in particular in the production of surfactants.

The targeted modulation of plant metabolic pathways by recombinant methods allows the modification of the plant metabolism in an advantageous manner which, when using traditional breeding methods, could only be achieved after a complicated procedure, if at all. Thus, unusual fatty acids, for example specific polyunsaturated fatty acids, are only synthesized in certain plants or not at all in plants and can therefore only be produced by expressing the relevant enzyme in transgenic plants [for example Millar et al. (2000) Trends Plant Sci 5:95-101].

Triacylglycerides and other lipids are synthesized from fatty acids. Fatty acid biosynthesis and triacylglyceride biosynthesis can be considered separate biosynthetic pathways owing to the compartmentalization, but as a single biosynthetic pathway in view of the end product. Lipid synthesis can be divided into two part-mechanisms, one which might be termed “prokaryotic” and another which might be termed “eukaryotic” (Browse et al. (1986) Biochemical J 235:25-31; Ohlrogge & Browse (1995) Plant Cell 7:957-970). The prokaryotic mechanism of the synthesis is localized in the plastids and comprises the biosynthesis of the free fatty acids which are exported into the cytosol, where they enter the eukaryotic mechanism in the form of fatty acid acyl-CoA esters and are esterified with glycerol 3-phosphate (G3P) to give phosphatidic acid (PA). PA is the starting point for the synthesis of neutral and polar lipids, The neutral lipids are synthesized on the endoplasmic reticulum via the Kennedy pathway, inter alia [Voelker (1996) Genetic Engineering, Setlow (ed.) 18:111-113; Shankline & Cahoon (1998) Annu Rev Plant Physiol Plant Mol Biol 49:611-649; Frentzen (1998) Lipids 100:161-166]. Besides the biosynthesis of triacylglycerides, G3P also plays a role in glycerol synthesis (for example for the purposes of osmoregulation and against low-temperature stress).

GP3, which is essential for the synthesis, is synthesized here by the reduction of dihydroxyacetone phosphate (DHAP) by means of glycerol 3-phosphate dehydrogenase (G3PDH), also termed dihydroxyacetone phosphate reductase. As a rule, NADH acts as reducing cosubstrate (EC 1.1.1.8). A further class of glycerol 3-phosphate dehydrogenases (EC 1.1.99.5) utilizes FAD as cosubstrate. The enzymes of this class catalyze the reaction of DHAP to G3PDH. In eukaryotic cells, the two classes of enzymes are distributed in different compartments, those which are NAD-dependent being localized in the cytosol and those which are FAD-dependent being localized in the mitochondria (for Saccharomyces cerevisiae, see, for example, Larsson et al., 1998, Yeast 14:347-357).

EP-A 0 353 049 describes an NAD-independent G3PDH from Bacillus sp. An NAD-independent G3PDH has also been identified in Saccharomyces cerevisiae [Miyata K, Nagahisa M (1969) Plant Cell Physiol 10 (3):635-643].

G3PDH is an essential enzyme in prokaryotes and eukaryotes which, besides having a function in lipid biosynthesis, is one of the enzymes responsible for maintaining the cellular redox status by acting on the NAD+/NADH ratio. Deletion of the GPD2 gene in Saccharomyces cerevisiae (one of two G3PDH isoforms in this yeast) results in reduced growth under anaerobic conditions. In addition, G3PDH appears to play a role in the stress response of yeast mainly to osmotic stress. Deletion of the GPD1 gene in Saccharomyces cerevisiae causes hypersensitivity to sodium chloride.

Sequences for G3PDHs have moreover been described for insects (Drosophila melanogaster, Drosophila virilis), plants (Arabidopsis thaliana, Cuphea lanceolata), mammals (Homo sapiens, Mus musculus, Sus scrofa, Rattus norvegicus), fish (Salmo salar, Osmerus mordax), birds (Ovis aries), amphibians (Xenopus laevis), nematodes (Caenorhabditis elegans), algae and bacteria.

Plant cells have at least two G3PDH isoforms, a cytoplasmic isoform and a plastidic isoform [Gee R W et al. (1988) Plant Physiol 86:98-103, Gee R W et al. (1988) Plant Physiol 87:379-383]. In plants, the enzymatic activity of glycerol 3-phosphate dehydrogenase was first found in potato tubors [Santora G T et al. (1979) Arch Biochem Biophys 196:403-411]. Further G3PDH activities which were localized in the cytosol and the plastids were detected in other plants such as peas, maize or soya [Gee R W et al. (1988) PLANT PHYSIOL 86(1): 98-103]. G3PDHs from algae such as, for example, two plastid G3PDH isoforms and one cytosolic G3PDH isoform from Dunaliella tertiolecta have furthermore been described [Gee R et al. (1993) Plant Physiol 103(1)243-249; Gee R et al. (1989) PLANT PHYSIOL 91(1):345-351]. As regards the plant G3PDH from Cuphea lanceolata, it has been proposed to obtain an increased oil content or a shift in the fatty acid pattern by overexpressing the enzyme, in plants (WO 95/06733). However, such effects have not been proven.

Bacterial G3PDHs and their function have been described [Hsu and Fox (1970) J Bacteriol 103:410-416 and Bell (1974) J Bacteriol 117:1065-1076].

WO 01/21820 describes the heterologous expression of a mutated E. coli G3PDH for increased stress tolerance and modification of the fatty acid composition in storage oils. The mutated E. coli G3PDH (gpsA2FR) exhibits a single amino acid substitution which brings about reduced inhibition via G3P. The heterologous expression of the gpsA2FR mutant leads to glycerolipids with an increased C16 fatty acid content and, accordingly, a reduced C18 fatty acid content. The modifications in the fatty acid pattern are relatively minor: an increase of 2 to 5% in the 16:0 fatty acids and of 1.5 to 3.5% in the 16:3 fatty acids, and a reduction in 18:2 and 18:3 fatty acids by 2 to 5% were observed. The total glycerolipid content remained unaffected.

WO 03/095655 describes the expression of the yeast protein Gpd1p in Arabidopsis. It was possible to increase the oil content of the Arabidopsis plants analyzed by approximately 22%. Individual seeds of a single transgenic line showed an increase by 41% in comparison with wild-type control plants. The disadvantage in this method is that Arabidopsis is a model plant which, owing its agronomic characteristics, is unsuitable for the commercial production of oils. Moreover, Arabidopsis accumulates significant amounts of eicosaenoic acid (20:1), which does not allow the oil to be used in foodstuffs or pharmaceuticals.

G3PDHs from yeasts (Ascomycetes) such as
a) Schizosaccharomyces pombe [Pidoux A L et al. (1990) Nucleic Acids Res 18 (23): 7145; GenBank Acc.-No.: X56162; Ohmiya R et al., (1995) Mol Microbiol 18(5):963-73; GenBank Acc.-No.: D50796, D50797],
b) Yarrowia lipolytica (GenBank Acc.-No.: AJ250328)
c) Zygosaccharomyces rouxii [Iwaki T et al. Yeast (2001) 18(8):737-44; GenBank Acc.-No: AB047394, AB047395, AB047397] or
d) Saccharomyces cerevisiae [Albertyn J et al. (1994) Mol Cell Biol 14(6):4135-44; Albertyn J et al. (1992) FEBS LETT 308(2):130-132; Merkel J R et al. (1982) Anal Biochem 122 (1):180-1185; Wang H T et al. (1994) J Bacteriol. 176(22):7091-5; Eriksson P et al. (1995) Mol Microbiol. 17(1):95-107; GenBank Acc.-No.: U04621, X76859, Z35169].
e) Emericella nidulans (GenBank Acc.-No.: AF228340)
f) Debaryomyces hansenii (GenBank Acc.-No.: AF210060)
   are furthermore described.

None of the methods described to date of increasing the oil content in transgenic plants leads to an increase in the oil content in cultivatable plants which is sufficient for a technical process. There is, therefore, still a great demand for increasing the total oil content in transgenic cultivatable plants, preferably in the seed of these plants. Such a method should meet the following criteria:
As few genes as possible should be introduced into the plant in order to increase the total oil content in the transgenic plants.
The method should be as simple and inexpensive as possible.
In order to achieve as high an oil yield as possible, plants with a high oil content should be employed.
A high oil yield should be achieved with the plants employed.
Saturated C₁₄-C₁₈-fatty acids should be present in the oil produced in as small amounts as possible.
The fatty acid profile should only be modified little, if possible not at all, between the wild type and the transgenic plant.
Furthermore, any bottlenecks in the precursors of oil biosynthesis or fatty acid biosynthesis should be eliminated in the method.

It was therefore an object to develop a method of increasing the total oil content in crop plants which features as many of the abovementioned properties as possible.

This object has been achieved by a method of increasing the total oil content in transgenic oil crop plants, wherein the transgenic oil crop plants comprise at least 20% by weight of oleic acid based on the total fatty acid content and which comprises the following method steps:
a) introducing into the oil crop plant, a nucleic acid sequence which codes for a glycerol 3-phosphate dehydrogenase from a yeast, and
b) expressing, in the oil crop plant, the glycerol 3-phosphate dehydrogenase encoded by the nucleic acid, and
c) selecting those oil crop plants in which the total oil content is increased by at least 25% by weight in the plant in comparison with the nontransgenic plant.

The transgenic oil crop plants advantageously comprise at least 21, 22, 23, 24 or 25% by Weight of oleic acid, advantageously at least 26, 27, 28, 29 or 30% by weight of oleic acid, based on the total fatty acid content, especially advantageously at least 35, 40, 45, 50, 55 or 60% by weight of oleic acid based on the total fatty acid content, very especially advantageously at least 61, 62, 63, 64, 65, 66, 67, 68, 69 or 70% by weight of oleic acid based on the total fatty acid content, or more. Plants which are advantageous for the method according to the invention furthermore have a preferred palmitic acid content of not more than 30, 29, 28, 27 or 26% by weight, advantageously of 25, 24, 23, 22, 21 or 20% by weight, especially advantageously of 15, 14, 13, 12, 11, 10 or 9% by weight, based on the total fatty acid content. Other advantageous plants have a linoleic acid content of at least 20, 25, 30, 35, 40, 45 or 50% by weight, advantageously 55, 60, 65 or 70% by weight, based on the total fatty acid content. Advantageous plants may also feature combination of the abovementioned fatty acids, the total fatty acid content being 100% by weight.

As a result of the method, the total oil content in the transgenic oil crop plants is increased by at least 26, 27, 28, 29 or 30% by weight, advantageously by at least 31, 32, 33, 34 or 35% by weight, especially advantageously by at least 36, 37, 38, 39 or 40% by weight, very especially advantageously by at least 41, 42, 43, 44 or 45% by weight.

Preferred oil crop plants used in the method have a high oil content in the seed. Advantageous plants have an oil content of at least 20, 25, 30, 35 or 40% by weight, advantageously of at least 41, 42, 43, 44 or 45% by weight, especially advantageously of at least 46, 47, 48, 49 or 50% by weight or more.

Oil crop plants which are preferred in the method produce oils, lipids and/or free fatty acids which comprise less than 4, 3, 2 or 1% by weight, advantageously less than 0.9; 0.8; 0.7; 0.6 or 0.5% by weight, especially advantageously less than 0.4; 0.3, 0.2; 0.1 or 0.09% by weight or less myristic acid. Further advantageous oil crop plants comprise less than 5, 4 or 3% by weight of palmitic acid and/or less than 2; 1.5 or 1% by weight of stearic acid.

Advantageous oil crop plants should not only have a high oil content in the seed, but also a low protein content in the seed. This protein content should, if possible, be less than 30, 25 or 20% by weight, advantageously less than 19, 18, 17, 16 or 15% by weight.

The oil crop plants which are preferred in the method advantageously feature no significant modification in the fatty acid profile of the C16:0, C16:3, C18:0, C18:1, C18:2, C18:3 and C20:0 fatty acids after the G3PDH-encoding nucleic acid sequences have been introduced, that is to say the relative percentages of the individual fatty acids which have been mentioned of the total fatty acid content in % by weight remain essentially the same. Essentially the same means that the variations in the percentages of the fatty acids vary by less than 5 percentage points.

Advantageous plants used in the method have a high oil yield per hectare. This oil yield is at least 100, 110, 120, 130, 140 or 150 kg oil/ha, advantageously at least 250, 300, 350, 400, 450 or 500 kg oil/ha, preferably at least 550, 600, 650, 700, 750, 800, 850, 900 or 950 kg oil/ha, especially preferably at least 1000 kg oil/ha, or more.

Plants which are suitable for the method according to the invention are, in principle, all cultivatable oil crop plants. Oil crop plants which are preferably employed for the method according to the invention are selected from the group of the plants consisting of the families Anacardiaceae, Arecaceae, Asteraceae, Brassicaceae, Cannabaceae, Euphorbiaceae, Fabaceae, Juglandaceae, Linaceae, Lythraceae, Oleaceae, Poaceae and Rosaceae which already naturally have a high oil content and/or which are already being employed for the industrial recovery of oils.

The plants employed in the method are especially advantageously selected from the group of the oil crop plants selected from the group consisting of the genera and species Anacardium occidentale, Arachis hypogaea, Borago officinalis, Brassica campestris, Brassica napus, Brassica rapa, Brassica juncea, Camelina sativa, Cannabis sativa, Carthamus tinctorius, Cocos nucifera, Crambe abyssinica, Cuphea ciliata, Elaeis guineensis, Glycine max, Gossypium hirsitum, Gossypium barbadense, Gossypium herbaceum, Helianthus annus, Linum usitatissimum, Oenothera biennis, Olea europaea, Ricinus communis, Zea mays, Juglans regia and Prunus dulcis, especially preferably among the genera and species Brassica campestris, Brassica napus, Brassica rapa, Brassica juncea, Camelina sativa, Helianthus annus, Linum usitatissimum and Carthamus tinctorius, very especially preferably Brassica campestris, Brassica napus, Brassica rapa, Brassica juncea and Camelina sativa.

In the present method, the seed-specific heterologous expression of the yeast gpd1p gene leads to a significant increase in the oil content as described above in the preferred plant family of the Brassicaceae, for example in Brassica napus and specifically in the seed. The increase in the oil content advantageously takes place to increase the triacylglycerides (reserve oils). In 3 independent lines, the oil content has been increased by approximately 35% in comparison with wild-type control plants (FIG. 4). The transgenic expression of the glycerol 3-phosphate dehydrogenase from yeast has advantageously shown no adverse effect on the growth or other properties of the transformed oil crop plants, such as the oil seed rape plants.

It has been possible to demonstrate that the increase in the content of, advantageously, triacylglycerides (reserve oils) is achieved by increasing the G3PDH activity. In the method according to the invention, it is not only the oil content but, advantageously, also the glycerol 3-phosphate content which is increased, advantageously in the maturing seed of the G3PDH-expressing oil crop plants, preferably of the transgenic Brassicaceae. Glycerol 3-phosphate is an important precursor in triacylglyceride biosynthesis and thus an essential precursor for increasing the oil content in oil crop plants, specifically in the seed.

For the purpose of the invention, the plants, or oil crop plants, include plant cells and certain tissues, organs and parts of plants, propagation material (such as seeds, tubers and fruits) or seed of plants, and plants in all their aspects such as anthers, fibers, root hairs, stems, leaves, embryos, calli, cotelydons, petioles, shoots, seedlings, crop material, plant tissue, reproductive tissue and cell cultures which is derived from the actual transgenic plant and/or can be used to bring about the transgenic plant. Mature plants are also included. Mature plants are understood as being plants at any developmental stage beyond the seedling. Seedling means a young, immature plant at an early developmental stage.

“Plant” comprises all annual and perennial monocotyledonous and dicotyledonous plants and includes the abovementioned advantageous oil crop plants.

Preferred monocotyledonous plants are selected in particular among the monocotyledonous crop plants such as, for example, the family Poaceae, such as maize.

In the method according to the invention, it is advantageous to use dicotyledonous oil crop plants. Preferred dicotyledonous plants are selected in particular among the dicotyledonous crop plants such as, for example,
Asteraceae such as sunflower, tagetes or calendula and others,
Brassicaceae, especially the genus Brassica, very particularly the species napus (oil seed rape), napus var. napus or rapa ssp. oleifera (canola), juncea (Indian mustard), Camelina sative (false flax) and others,
Leguminosae, especially the genus Glycine, very especially the species max (soybean) soya or peanut and others
   and linseed, soya, cotton or hemp.

Transgenic plants with an increased oil content can be marketed directly without isolation of the synthesized oil being necessary, In the method according to the invention, plants are to be understood as meaning whole plants and also all plant parts, plant organs or plant parts such as leaf, stem, seed, root, tuber, anthers, fibers, root hairs, stalks, embryos, calli, cotelydons, petioles, crop material, plant tissue, reproductive tissue, cell cultures which are derived from the transgenic plant and/or which can be used to bring about the transgenic plant. The seed includes all parts of the seed such as seed coats, epidermal cells and seed cells, endosperm or embryonic tissue. However, the oils produced by the method according to the invention can also be isolated from the plants in the form of their oils, fat, lipids and/or free fatty acids. Oils produced by the method can be obtained by harvesting the plants either from the culture in which they grow or from the field. This can be affected by pressing or extracting the plant parts, preferably the seeds of the plants. Here, the oils can be obtained by pressing by “cold beating or cold pressing” without input of heat. The plant parts, specifically the seeds, are comminuted, steam-treated or roasted beforehand so that they can be digested more easily; The seeds pretreated in this manner can then be pressed or extracted with solvents, such as warm hexane. Thereafter, the solvent is removed again. In this manner, more than 96% of the oils produced by the method can be isolated. The products thus obtained are then processed further, i.e. refined. Here, initially, the plant mucilage and matter causing turbidity are removed. What is known as desliming can be affected enzymatically or, for example, chemico-physically by addition of acid such as phosphoric acid. Thereafter, the free fatty acids may be removed by treatment with a base, for example sodium hydroxide solution. To remove the alkali still present in the product, the product obtained is washed thoroughly with water and dried. To remove the pigments which are still present in the product, the products are subjected to bleaching with, for example, bleaching earth or activated carbon. Finally, the product is deodorized using, for example, steam.

One embodiment according to the invention is the use of the oils prepared by the method according to the invention or obtained by mixing these oils with animal, microbial or vegetable oils, lipids or fatty acids in feeds, foodstuffs, cosmetics or pharmaceuticals. The oils prepared by the method according to the invention can be used in a manner known to the person skilled in the art for mixing with other oils, lipids, fatty acids or fatty acid mixtures of animal origin, such as, for example, fish oils. The fatty acids present in the oils prepared in accordance with the invention, which were liberated from the oils by treatment with base, can also be added in a customary amount to foodstuffs, feedstuffs, cosmetics and/or pharmaceuticals, either directly or after mixing with other oils, lipids, fatty acids or fatty acid mixtures of animal origin such as, for example, fish oils.

The oils prepared by the method comprise compounds such as sphingolipids, phosphoglycerides, lipids, glycolipids, phospholipids, monoacylglycerides, diacylglycerides, triacylglycerides or other fatty acid esters, preferably triacylglycerides (see Table 1).

From the oils thus prepared by the method according to the invention, the saturated and unsaturated fatty acids which are present therein can be liberated for example by treatment with alkali, for example with aqueous KOH or NaOH, or by acidic hydrolysis, advantageously in the presence of an alcohol such as methanol or ethanol, or via enzymatic cleavage, and isolated for example by phase separation and subsequent acidification using, for example, H₂SO₄. The fatty acids can also be liberated directly without the above-described work-up.

The term “oil” is also understood to include “lipids” or “fats” or “fatty acid mixtures”, which comprise unsaturated, saturated, preferably esterified, fatty acid(s), preferably bound to triglycerides. It is preferred for the oil. The oil may comprise various other saturated or unsaturated fatty acids, such as, for example, palmitic acid, palmitoleic acid, stearic acid, oleic acid, linoleic acid or a-linolenic acid, and the like. In particular, the content of the various fatty acids in the oil may vary, depending on the original plant.

“Total oil content” is understood as meaning the sum of all oils, lipids, fats or fatty acid mixtures, preferably the sum of all triacylglycerides.

“Oils” comprises neutral and/or polar lipids and mixtures of these. Those mentioned in table 1 may be mentioned by way of example, but not by limitation.

TABLE 1
Classes of plant lipids

| | |
|---|---|
| Neutral lipids | Triacylglycerol (TAG) |
| | Diacylglycerol (DAG) |
| | Monoacylglycerol (MAG) |
| Polar lipids | Monogalactosyldiacylglycerol (MGDG) |
| | Digalactosyldiacylglycerol (DGDG) |
| | Phosphatidylglycerol (PG) |
| | Phosphatidylcholine (PC) |
| | Phosphatidylethanolamine (PE) |
| | Phosphatidylinositol (PI) |
| | Phosphatidylserine (PS) |
| | Sulfoquinovosyldiacylglycerol (SQD) |

Neutral lipids preferably refers to triacylglycerides. Both neutral and polar lipids may comprise a wide range of various fatty acids. The fatty acids mentioned in table 2 may be mentioned by way of example, but not by limitation.

TABLE 2
Overview over various fatty acids (selection)

| Nomenclature ¹ | Name |
|---|---|
| 14:0 | Myristic acid |
| 16:0 | Palmitic acid |
| 16:1 | Palmitoleic acid |
| 16:3 | Roughanic acid |
| 18:0 | Stearic acid |
| 18:1 | Oleic acid |
| 18:2 | Linoleic acid |
| α-18:3 | Linolenic acid |
| γ-18:3 | Gamma-linolenic acid ⁺ |
| 20:0 | Arachidic acid |
| 20:1 | Eicosaenoic acid |
| 22:6 | Docosahexaenoic acid (DHA) * |
| 20:2 | Eicosadienoic acid |
| 20:4 | Arachidonic acid (AA) ⁺ |
| 20:5 | Eicosapentaenoic acid (EPA) ⁺ |
| 22:1 | Erucic acid |

¹ chain length: number of double bonds
⁺ occurring only in very few plant genera
* not naturally occurring in higher plants

Oils preferably means seed oils.

“Increasing” the total oil content means increasing the oil content in a plant or in a part, tissue or organ thereof, preferably in the seed organs of the plant. In this context, the oil content is increased by at least 25%, preferably at least 30%, especially preferably at least 35%, very especially preferably at least 40%, most preferably at least 45% or more in comparison with a starting plant which is not subjected to the method according to the invention, but otherwise unmodified, and under otherwise identical conditions. Conditions in this context means all of the conditions which are relevant for germination, culture or growth of the plant such as soil conditions, climatic conditions, light conditions, fertilization, irrigation, plant protection treatments and the like.

Increasing the content of glycerol 3-phosphate in an oil crop plant is understood as meaning increasing the content in a plant or in a part of the plant, in tissues or in organs of the same, preferably in the seed of the plant. Here, the glycerol 3-phosphate content is increased by at least 25, 30, 35, 40, 45 or 50% by weight, preferably by at least 60, 70, 80, 90 or 100%, especially preferably by at least 110, 120, 130, 140 or 150%, very especially preferably by at least 200, 250 or 300%, most preferably by at least 350 or 400% or more in comparison with an original plant which has not been subjected to the method according to the invention, but is otherwise unmodified, under otherwise identical conditions, Conditions in this context means all of the conditions which are relevant for germination, culture or growth of the plant such as soil conditions, climatic conditions, light conditions, fertilization, irrigation, plant protection treatments and the like.

“Yeast glycerol 3-phosphate dehydrogenase” (termed yeast “G3PDH” hereinbelow) generally refers to all those enzymes which are capable of converting dihydroxyacetone phosphate (DHAP) into glycerol 3-phosphate (G3P)—preferably using a cosubstrate such as NADH or NADPH—and which are naturally expressed in a yeast.

Yeast refers to the group of unicellular fungi with a pronounced cell wall and formation of a pseudomycelium (in contrast to molds). They reproduce vegetatively by budding and/or fission (Schizosaccharomyces and Saccharomycodes, respectively).

Encompassed are what are known as false yeasts, preferably the families Cryptococcaceae, Sporobolomycetaceae with the genera Cryptococcus, Torulopsis, Pityrosporum, Brettanomyces, Candida, Kloeckera, Trigonopsis, Trichosporon, Rhodotorula and Sporobolomyces and Bullera, and true yeasts (yeasts which also reproduce generatively; ascus), preferably the families Endo- and Saccharomycetaceae, with the genera Saccharomyces, Debaromyces, Lipomyces, Hansenula, Endomycopsis, Pichia, Hanseniaspora. Most preferred are the genera Saccharomyces cerevisiae, Pichia pastoris, Hansenula polymorpha, Schizosaccharomyces pombe, Kluyveromyces lactis, Zygosaccharomyces rouxii, Yarrowia lipolitica, Emericella nidulans, Aspergillus nidulans, Deparymyces hansenii and Torulaspora hansenii.

Yeast G3PDH means, in particular, polypeptides which have the following characteristics as “essential characteristics”:

| | |
|---|---|
| a) | the conversion of dihydroxyacetone phosphate |
| | into glycerol 3-phosphate using NADH as |
| | cosubstrate (EC 1.1.1.8), and |
| b) | a peptide sequence comprising at least one |
| | sequence motif selected from the group of |
| | sequence motifs consisting of |

| | | |
|---|---|---|
| c) | GSGNWGT(A/T)IAK | (SEQ ID NO: 22) |
| d) | CG(V/A)LSGAN(L/I/V)AXE(V/I)A | (SEQ ID NO: 26) |
| e) | (L/V)FXRPYFXV | (SEQ ID NO : 27) |

preferred is the sequence motif selected from the
group consisting of

| | | |
|---|---|---|
| f) | GSGNWGTTIAKV(V/I)AEN | (SEQ ID NO : 29) |
| g) | NT(K/R)HQNVKYLP | (SEQ ID NO: 30) |
| h) | D(I/V)LVFN(I/V)PHQFL | (SEQ ID NO: 31) |
| i) | RA(I/V)SCLKGFE | (SEQ ID NO: 32) |
| j) | CGALSGANLA(P/T)EVA | (SEQ ID NO: 33) |
| K) | LFHRPYFHV | (SEQ ID NO: 34) |
| I) | GLGEII(K/R)FG | (SEQ ID NO: 35) |

The peptide sequence particularly preferably comprises at least 2 or 3, very particularly preferably at least 4 or 5, most preferably all of the sequence motifs selected from the group of the sequence motifs i), ii) and iii) or selected from the group of the sequence motifs iv), v), vi), vii), viii), ix) and x). (Terms in brackets refer to amino acids which are possible at this position as alternatives, for example (V/I) means that valin or isoleucin are possible at this position. The sequence listings only mention one of the possible variants in each case).

Furthermore, a yeast G3PDH may optionally—in addition to at least one of the abovementioned sequence motifs i) to x)—comprise further sequence motifs selected from the group consisting of

| | | |
|---|---|---|
| m) | H(E/Q)NVKYL | (SEQ ID NO: 23) |
| n) | (D/N)(I/V)(L/I)V(F/W)(V/N)(L/I/ | (SEQ ID NO: 24) |
| | V)PHQF)(V/L/I) | |
| o) | (A/G)(I/V)SC(L/I)KG | (SEQ ID NO: 25) |
| p) | G(L/M)(L/G)E(M/I)(I/Q)(R/K/N)F | (SEQ ID NO: 28) |
| | (G/S/A) | |

Most preferably, yeast G3PDH means the yeast protein Gpd1p as shown in SEQ ID NO: 2, and functional equivalents thereof, as well as functionally equivalent portions of the above. Functionally equivalent portions are understood as meaning sequences which are at least 51, 60, 90 or 120 bp, advantageously at least 210, 300, 330, 420 or 450 bp, especially advantageously at least 525, 540, 570 or 600 bp, very especially advantageously at least 660, 720, 810, 900 or 1101 bp or more in length.

Functional equivalents means, in particular, natural or artificial mutations of the yeast protein Gpd1p as shown in SEQ ID NO: 2 and homologous polypeptides from other yeasts which have essentially the same characteristics of a yeast G3PDH as defined above. Mutations comprise substitutions, additions, deletions, inversion or insertions of one or more amino acid residues. Especially preferred are the polypeptides described by SEQ ID NO: 4, SEQ ID NO: 5, SEQ ID NO: 7, SEQ ID NO: 9, SEQ ID NO: 11, SEQ ID NO: 12, SEQ ID NO: 14, SEQ ID NO: 16, SEQ ID NO: 38 or SEQ ID NO: 40.

The yeast G3PDHs to be employed advantageously within the scope of the present invention can be found readily by database searches or by screening gene or cDNA libraries using the yeast G3PDH sequence shown in SEQ ID NO: 2, which is given by way of example, or the nucleic acid sequence as shown in SEQ ID NO: 1, which encodes the latter, as search sequence or probe.

Said functional equivalents preferably have at least 50 or 60%, especially preferably at least 70 or 80%, especially preferably at least 85 or 90%, most preferably at least 91, 92, 93, 94, 95 or 96% or more homology with the protein with the SEQ ID NO: 2.

Homology between two polypeptides is understood as meaning the identity of the amino acid sequence over the entire sequence length which is calculated by comparison with the aid of the program algorithm GAP (Wisconsin Package Version 10.0, University of Wisconsin, Genetics Computer Group (GCG), Madison, USA), setting the following parameters:

| | |
|---|---|
| Gap Weight: 8 | Length Weight: 2 |
| Average Match: 2,912 | Average Mismatch: −2,003 |

For example, a sequence with at least 80% homology with the sequence SEQ ID NO: 2 at the protein level is understood as meaning a sequence which, upon comparison with the sequence SEQ ID NO: 2 within the above program algorithm and the above parameter set has at least 80% homology.

Functional equivalents also comprises those proteins which are encoded by nucleic acid sequences which have at least 60, 70 or 80%, especially preferably at least 85, 87, 88, 89 or 90%, especially preferably at least 91, 92, 93, 94 or 95%, most preferably at least 96, 97, 98 or 99% homology with the nucleic acid sequence with the SEQ ID NO: 1.

Homology between two nucleic acid sequences is understood as meaning the identity of the two nucleic acid sequences over the respective entire sequence length which is calculated by comparison with the aid of the program algorithm GAP (Wisconsin Package Version 10.0, University of Wisconsin, Genetics Computer Group (GCG), Madison, USA; Altschul et al. (1997) Nucleic Acids Res. 25:3389 et seq.), setting the following parameters:

| | |
|---|---|
| Gap Weight: 50 | Length Weight: 3 |
| Average Match: 10 | Average Mismatch: 0 |

For example, a sequence which has at least 80% homology with the sequence SEQ ID NO: 1 at the nucleic acid level is understood as meaning a sequence which, upon comparison with the sequence SEQ ID NO: 1 within the above program algorithm with the above parameter set has a homology of at least 80%.

Functional equivalents also comprises those proteins which are encoded by nucleic acid sequences which hybridize under standard conditions with a nucleic acid sequence described by SEQ ID NO. 1, the nucleic acid sequence which is complementary thereto or parts of the above and which have the essential characteristics for a yeast G3PDH.

“Standard hybridization conditions” is to be understood in the broad sense and means both stringent and less stringent hybridization conditions. Such hybridization conditions are described, for example, by Sam brook J, Fritsch E F, Maniatis T et al., in Molecular Cloning (A Laboratory Manual), 2nd edition, Cold Spring Harbor Laboratory Press, 1989, pages 9.31-9.57) or in Current Protocols in Molecular Biology, John Wiley & Sons, N.Y. (1989), 6.3.1-6.3.6.

For example, the conditions during the wash step can be selected from the range of low-stringency conditions (with approximately 2×SSC at 50° C.) and high-stringency conditions (with approximately 0.2×SSC at 50° C., preferably at 65° C.) (20×SSC: 0.3 M sodium citrate, 3 M NaCl, pH 7.0). Denaturing agents such as, for example, formamide or SDS may also be employed during hybridization, In the presence of 50% formamide, hybridization is preferably carried out at 42° C.

In the method according to the invention, the nucleic acid sequences used are advantageously introduced into a transgenic expression construct which can ensure a transgenic expression of a yeast G3PDH in a plant or a tissue, organ, part, cell or propagation material of the plant.

In the expression constructs, a nucleic acid molecule coding for a yeast G3PDH is preferably in operable linkage with at least one genetic control element (for example a promoter and/or a terminator) which ensures expression in a plant organism or a tissue, organ, part, cell or propagation material of same.

Transgenic expression cassettes which are especially preferably used are those which comprise a nucleic acid sequence coding for a glycerol 3-phosphate dehydrogenase which is selected from the group of the sequences consisting of
a) a sequence with SEQ ID NO: 1, SEQ ID NO: 3, SEQ ID NO: 6, SEQ ID NO: 8, SEQ ID NO: 10, SEQ ID NO: 13, SEQ ID NO: 15, SEQ ID NO: 37 or SEQ ID NO: 39 or
b) a sequence which, in accordance with the degeneracy of the genetic code, is derived from a sequence with the sequence shown in SEQ ID NO: 1, SEQ ID NO: 3, SEQ ID NO: 6, SEQ ID NO: 8, SEQ ID NO: 10, SEQ ID NO: 13, SEQ ID NO: 15, SEQ ID NO: 37 or SEQ ID NO: 39, or
   a sequence which has at least 60% identity with the sequence with SEQ ID NO: 1.

Operable linkage is understood as meaning, for example, the sequential arrangement of a promoter with the nucleic acid sequence coding for a yeast G3PDH which is to be expressed (for example the sequence as shown in SEQ ID NO: 1) and, if appropriate, further regulatory elements such as, for example, a terminator in such a way that each of the regulatory elements can fulfil its function when the nucleic acid sequence is expressed recombinantly. Direct linkage in the chemical sense is not necessarily required for this purpose. Genetic control sequences such as, for example, enhancer sequences can also exert their function on the target sequence from positions which are further removed, or indeed from other DNA molecules. Preferred arrangements are those in which the nucleic acid sequence to be expressed recombinantly is positioned behind the sequence acting as promoter so that the two sequences are linked covalently to each other. The distance between the promoter sequence and the nucleic acid sequence to be expressed recombinantly is preferably less than 200 base pairs, especially preferably less than 100 base pairs, very especially preferably less than 50 base pairs.

Operable linkage and a transgenic expression cassette can both be produced by means of conventional recombination and cloning techniques as they are described, for example, in Maniatis T, Fritsch E F and Sambrook J (1989) Molecular Cloning: A Laboratory Manual, Cold Spring Harbor Laboratory, Cold Spring Harbor (NY), in Silhavy T J, Berman M L und Enquist L W (1984) Experiments with Gene Fusions, Cold Spring Harbor Laboratory, Cold Spring Harbor (NY), in Ausubel F M et al. (1987) Current Protocols in Molecular Biology, Greene Publishing Assoc. and Wiley Interscience and in Gelvin et al. (1990) In: Plant Molecular Biology Manual. However, further sequences which, for example, act as a linker with specific cleavage sites for restriction enzymes, or as a signal peptide, may also be positioned between the two sequences. Also, the insertion of sequences may lead to the expression of fusion proteins. Preferably, the expression cassette composed of a promoter linked to a nucleic acid sequence to be expressed can be in a vector-integrated form and can be inserted into a plant genome for example by transformation.

However, an expression cassette is also understood as meaning those constructs where the nucleic acid sequence coding for a yeast G3PDH is placed behind an endogenous promoter in such a way that the latter brings about the expression of the yeast G3PDH.

Promoters which are preferably introduced into the transgenic expression cassettes are those which are operable in a plant organism or a tissue, organ, part, cell or propagation material of same. Promoters which are operable in plant organisms is understood as meaning in principle any promoter which is capable of governing the expression of genes, in particular heterologous genes, in plants or plant parts, plant cells, plant tissues or plant cultures. In this context, expression may be, for example, constitutive, inducible or development-dependent.

The following are preferred:

### a) Constitutive Promoters

“Constitutive” promoters refers to those promoters which ensure expression in a large number of, preferably all, tissues over a substantial, period of plant development, preferably at all times during plant development (Benfey et al. (1989) EMBO J. 8:2195-2202). A plant promoter or promoter originating from a plant virus is especially preferably used. The promoter of the CaMV (cauliflower mosaic virus) 35S transcript (Franck et al. (1980) Cell 21:285-294; Odell et al. (1985) Nature 313:810-812; Shewmaker et al. (1985) Virology 140:281-288; Gardner et al. (1986) Plant Mol Biol 6:221-228) or the 19S CaMV promoter (U.S. Pat. No. 5,352,605; WO 84/02913; Benfey et al. (1989) EMBO J. 8:2195-2202) are especially preferred. Another suitable constitutive promoter is the Rubisco small subunit (SSU) promoter (U.S. Pat. No. 4,962,028), the leguminB promoter (GenBank Acc. No. X03677), the promoter of nopaline synthase from Agrobacterium, the TR dual promoter, the OCS (octopine synthase) promoter from Agrobacterium, the ubiquitin promoter (Holtorf S et al. (1995) Plant Mol Biol 29:637-649), the ubiquitin 1 promoter (Christensen et al. (1992) Plant Mol Biol 18:675-689; Bruce et al. (1989) Proc Natl Acad Sci USA 86:9692-9696), the Smas promoter, the cinnamyl alcohol dehydrogenase promoter (U.S. Pat. No. 5,683,439), the promoters of the vacuolar ATPase subunits, the promoter of the Arabidopsis thaliana nitrilase-1 gene (GenBank Acc. No.: U38846, nucleotides 3862 to 5325 or else 5342) or the promoter of a proline-rich protein from wheat (WO 91/13991), and further promoters of genes whose constitutive expression in plants is known to the skilled worker. The CaMV 35S promoter and the Arabidopsis thaliana nitrilase-1 promoter are preferred.

### b) Tissue-Specific Promoters

Furthermore preferred are promoters with specificities for seeds, such as, for example, the phaseolin promoter (U.S. Pat. No. 5,504,200; Bustos M M et al. (1989) Plant Cell 1(9):839-53), the promoter of the 2S albumin gene (Joseffson L G et al. (1987) J Biol Chem 262:12196-12201), the legumin promoter (Shirsat A et al. (1989) Mol Gen Genet. 215(2):326-331), the USP (unknown seed protein) promoter (Bäumlein H et al. (1991) Mol Gen Genet. 225(3):459-67), the napin gene promoter (U.S. Pat. No. 5,608,152; Stalberg K et al. (1996) L Planta 199:515-519), the promoter of the sucrose binding protein (WO 00/26388) or the legumin B4 promoter (LeB4; Bäumlein H et al. (1991) Mol Gen Genet. 225: 121-128; Bäumlein et al. (1992) Plant Journal 2(2).233-9; Fiedler U et al. (1995) Biotechnology (NY) 13(10):1090f), the oleosin promoter from Arabidopsis (WO 98/45461), and the Bce4 promoter from Brassica (WO 91/13980).
Further suitable seed-specific promoters are those of the genes coding for high-molecular weight glutenin (HMWG), gliadin, branching enzyme, ADP glucose pyrophosphatase (ASPase) or starch synthase. Promoters which are furthermore preferred are those which permit seed-specific expression in monocots such as maize, barley, wheat, rye, rice and the like. The promoter of the Ipt2 or Ipt1 gene (WO 95/15389, WO 95/23230) or the promoters described in WO 99/16890 (promoters of the hordein gene, the glutelin gene, the oryzin gene, the prolamin gene, the gliadin gene, the glutelin gene, the zein gene, the casirin gene or the secalin gene) can advantageously be employed.

### c) Chemically Inducible Promoters

The expression cassettes may also comprise a chemically inducible promoter (review article: Gatz et al. (1997) Annu Rev Plant Physiol Plant Mol Biol 48:89-108), by means of which the expression of the exogenous gene in the plant can be controlled at a particular point in time. Such promoters such as, for example, the PRP1 promoter (Ward et al. (1993) Plant Mol Biol 22:361-366), a salicylic acid-inducible promoter (WO 95/19443), a benzenesulfonamide-inducible promoter (EP 0 388 186), a tetracyclin-inducible promoter (Gatz et al. (1992) Plant J 2:397-404), an abscisic-acid-inducible promoter (EP 0 335 528) or an ethanol- or cyclohexanone-inducible promoter (WO 93/21334) can likewise be used. Also suitable is the promoter of the glutathione-S transferase isoform II gene (GST-II-27), which can be activated by exogenously applied safeners such as, for example, N,N-diallyl-2,2-dichloroacetamide (WO 93/01294) and which is operable in a large number of tissues of both monocots and dicots.

Especially preferred are constitutive promoters and very especially preferred seed-specific promoters, in particular the napin promoter and the USP promoter.

In addition, further promoters which make possible expression in further plant tissues or in other organisms such as, for example, E. coli bacteria, may be linked operably with the nucleic acid sequence to be expressed. Suitable plant promoters are, in principle, all of the above-described promoters.

The nucleic acid sequences present in the transgenic expression cassettes or vectors can be linked operably with further genetic control sequences besides a promoter. The term genetic control sequences is to be understood in the broad sense and refers to all those sequences which have an effect on the establishment or the function of the expression cassette according to the invention. Genetic control sequences modify, for example, transcription and translation in prokaryotic or eukaryotic organisms. The expression cassettes according to the invention preferably comprise a plant-specific promoter 5′-upstream of the nucleic acid sequence to be expressed recombinantly in each case and, as additional genetic control sequence, a terminator sequence 3′-downstream, and, if appropriate, further customary regulatory elements, in each case linked operably with the nucleic acid sequence to be expressed recombinantly.

Genetic control sequences also comprise further promoters, promoter elements or minimal promoters capable of modifying the expression-controlling properties. Thus, genetic control sequences can, for example, bring about tissue-specific expression which is additionally dependent on certain stress factors. Such elements are, for example, described for water stress, abscisic acid (Lam E and Chua N H, J Biol Chem 1991; 266(26): 17131-17135) and thermal stress (Schoffl F et al., (1989) Mol Gen Genetics 217(2-3):246-53).

Further advantageous control sequences are, for example, in the Gram-positive promoters amy and SPO2, and in the yeast or fungal promoters ADC1, MFa, AC, P-60, CYC1, GAPDH, TEF, rp28, ADH.

In principle, all natural promoters with their regulatory sequences like those mentioned above may be used for the method according to the invention. In addition, synthetic promoters may also be used advantageously.

Genetic control sequences further also comprise the 5′-untranslated regions, introns or nonencoding 3′-region of genes, such as, for example, the actin-1 intron, or the Adh1-S introns 1, 2 and 6 (for general reference, see: The Maize Handbook, Chapter 116, Freeling and Walbot, Eds., Springer, New York (1994)). It has been demonstrated that these may play a significant role in regulating gene expression. Thus, it has been demonstrated that 5′-untranslated sequences can enhance the transient expression of heterologous genes. Translation enhancers which may be mentioned by way of example are the tobacco mosaic virus 5′ leader sequence (Gallie et al., (1987) Nucl Acids Res 15:8693-8711) and the like. They may furthermore promote tissue specificity (Rouster J et al. (1998) Plant J 15:435-440).

The expression cassette can advantageously comprise one or more of what are known as enhancer sequences in operable linkage with the promoter, and these make possible an increased recombinant expression of the nucleic acid sequence. Additional advantageous sequences such as further regulatory elements or terminators may also be inserted at the 3′ end of the nucleic acid sequences to be expressed recombinantly. One or more copies of the nucleic acid sequences to be expressed recombinantly may be present in the gene construct.

Polyadenylation signals which are suitable as control sequences are plant polyadenylation signals, preferably those which correspond essentially to Agrobacterium tumefaciens T-DNA polyadenylation signals, in particular those of gene 3 of the T-DNA (octopine synthase) of the Ti plasmid pTiACHS (Gielen et al. (1984) EMBO J. 3:835 et seq.) or functional equivalents thereof. Examples of especially suitable terminator sequences are the OCS (octopine synthase) terminator and the NOS (nopaline synthase) terminator.

Control sequences are furthermore understood as those which make possible homologous recombination or insertion into the genome of a host organism, or removal from the genome. In the case of homologous recombination, for example, the coding sequence of a specific endogenous gene can be exchanged in a directed fashion for the sequence encoding a dsRNA. Methods such as the cre/lox technology permit a tissue-specific, possibly inducible, removal of the expression cassette from the genome of the host organism (Sauer B (1998) Methods. 14(4):381-92). Here, certain flanking sequences are added to the target gene (lox sequences), and these make possible removal by means of cre recombinase at a later point in time.

A expression cassette and the vectors derived from it may comprise further functional elements. The term functional element is to be understood in the broad sense and refers to all those elements which have an effect on generation, replication or function of the expression cassettes, vectors or transgenic organisms according to the invention. Examples which may be mentioned, but not by way of limitation, are:
a) Selection markers which confer resistance to a metabolism inhibitor such as 2-deoxyglucose 6-phosphate (WO 98/45456), antibiotics or biocides, preferably herbicides, such as, for example, kanamycin, G 418, bleomycin, hygromycin, or phosphinothricin and the like. Particularly preferred selection markers are those which confer resistance to herbicides. The following may be mentioned by way of example: DNA sequences which encode phosphinothricin acetyltransferases (PAT) and which inactivate glutamine synthase inhibitors (bar and pat gene), 5-enolpyruvylshikimate 3-phosphate synthase genes (EPSP synthase genes), which confer resistance to Glyphosate® (N-(phosphonomethyl)glycine), the gox gene, which encodes Glyphosate®-degrading enzyme (Glyphosate oxidoreductase), the deh gene (encoding a dehalogenase which inactivates dalapon), sulfonylurea- and imidazolinone-inactivating acetolactate synthases, and bxn genes which encode nitrilase enzymes which degrade bromoxynil, the aasa gene, which confers resistance to the antibiotic apectinomycin, the streptomycin phosphotransferase (SPT) gene, which permits resistance to streptomycin, the neomycin phosphotransferase (NPTII) gene, which confers resistance to kanamycin or geneticidin, the hygromycin phosphotransferase (HPT) gene, which confers resistance to hygromycin, the acetolactate synthase gene (ALS), which confers resistance to sulfonylurea herbicides (for example mutated ALS variants with, for example, the S4 and/or Hra mutation).
b) Reporter genes which encode readily quantifiable proteins and which allow the transformation efficacy or the expression site or time to be assessed via their intrinsic color or enzyme activity. Very particularly preferred in this context are reporter proteins (Schenborn E, Groskreutz D. Mol. Biotechnol. 1999, 13(1):29-44) such as the “green fluorescence protein” (GFP) (Sheen et al. (1995) Plant Journal 8(5):777-784), chloramphenicol transferase, a luciferase (Ow et al. (1986) Science 234:856-859), the aequorin gene (Prasher et al. (1985) Biochem Biophys Res Commun 126(3):1259-1268), β-galactosidase, with β-glucuronidase being very particularly preferred (Jefferson et al. (1987) EMBO J. 6:3901-3907).
c) Replication origins which allow replication of the expression cassettes or vectors according to the invention in, for example, E. coli. Examples which may be mentioned are ORI (origin of DNA replication), the pBR322 ori or the P15A ori (Sambrook et al.: Molecular Cloning. A Laboratory Manual, 2nd ed. Cold Spring Harbor Laboratory Press, Cold Spring Harbor, N.Y., 1989).
d) Elements which are required for agrobacterium-mediated plant transformation such as, for example, the right or left border of the T-DNA, or the vir region.

To select cells which have successfully undergone homologous recombination or else cells which have successfully been transformed, it is generally required additionally to introduce a selectable marker which confers resistance to a biocide (for example a herbicide), a metabolism inhibitor such as 2-deoxyglucose 6-phosphate (WO 98/45456) or an antibiotic to the cells which have successfully undergone recombination. The selection marker permits the selection of the transformed cells from untransformed cells (McCormick et al. (1986) Plant Cell Reports 5:81-84).

In addition, the recombinant expression cassette or the expression vectors may comprise further nucleic acid sequences which do not code for a yeast G3PDH and whose recombinant expression leads to a further increase in fatty acid biosynthesis (as a consequence of proOIL). By way of example, but not by limitation, this proOIL nucleic acid sequence which is additionally expressed recombinantly can be selected from among nucleic acids encoding acetyl-CoA carboxylase (ACCase), glycerol 3-phosphate acyltransferase (GPAT), lysophosphatidate acyltransferase (LPAT), diacylglycerol acyltransferase (DAGAT) and phospholipid:diacylglycerol acyltransferase (PDAT). Such sequences are known to the skilled worker and are readily accessible from databases or suitable cDNA libraries of the respective plants.

An expression cassette according to the invention can advantageously be introduced into an organism or cells, tissues, organs, parts or seeds thereof (preferably into plants or plant cells, tissues, organs, parts or seeds) by using vectors in which the expression cassettes are present. The invention therefore furthermore relates to said recombinant vectors which comprise a recombinant expression cassette for a yeast G3PDH.

For example, vectors may be plasmids, cosmids, phages, viruses or else agrobacteria. The expression cassette can be introduced into the vector (preferably a plasmid vector) via a suitable restriction cleavage site. The resulting vector is first introduced into E. coli. Correctly transformed E. coli are selected, grown, and the recombinant vector is obtained with methods known to the skilled worker. Restriction analysis and sequencing may be used for verifying the cloning step. Preferred vectors are those which make possible stable integration of the expression cassette into the host genome.

Such a transgenic plant organism is generated, for example, by means of transformation or transfection by means of the corresponding proteins or nucleic acids. The generation of a transformed organism (or a transformed cell or tissue) requires introducing the DNA in question (for example the expression vector), RNA or protein into the host cell in question. A multiplicity of methods are available for this procedure, which is termed transformation (or transduction or transfection) (Keown et al. (1990) Methods in Enzymology 185:527-537). Thus, the DNA or RNA can be introduced for example directly by microinjection or by bombardment with DNA-coated microparticles. The cell may also be permeabilized chemically, for example with polyethylene glycol, so that the DNA may reach the cell by diffusion. The DNA can also take place by protoplast fusion with other DNA-comprising units such as minicells, cells, lysosomes or liposomes. Electroporation is a further suitable method for introducing DNA; here, the cells are permeabilized reversibly by an electrical pulse. Soaking plant parts in DNA solutions, and pollen or pollen tube transformation, are also possible. Such methods have been described (for example in Bilang et al. (1991) Gene 100:247-250; Scheid et al. (1991) Mol Gen Genet. 228:104-112; Guerche et al. (1987) Plant Science 52:111-116; Neuhause et al. (1987) Theor Appl Genet. 75:30-36; Klein et al. (1987) Nature 327:70-73; Howell et al. (1980) Science 208:1265; Horsch et al. (1985) Science 227:1229-1231; DeBlock et al. (1989) Plant Physiology 91:694-701; Methods for Plant Molecular Biology (Weissbach and Weissbach, eds.) Academic Press Inc. (1988); and Methods in Plant Molecular Biology (Schuler and Zielinski, eds.) Academic Press Inc. (1989)).

In plants, the methods which have been described for transforming and regenerating plants from plant tissues or plant cells are exploited for transient or stable transformation. Suitable methods are, in particular, protoplast transformation by polyethylene glycol-induced DNA uptake, the biolistic method with the gene gun, what is known as the particle bombardment method, electroporation, the incubation of dry embryos in DNA-containing solution, and microinjection.

In addition to these “direct” transformation techniques, transformation may also be effected by bacterial infection by means of Agrobacterium tumefaciens or Agrobacterium rhizogenes and the transfer of corresponding recombinant Ti plasmids or Ri plasmids by infection with transgenic plant viruses. Agrobacterium-mediated transformation is best suited to cells of dicotyledonous plants. The methods are described, for example, in Horsch R B et al. (1985) Science 225: 1229f).

When agrobacteria are used, the expression cassette is to be integrated into specific plasmids, either into a shuttle, or intermediate, vector or into a binary vector. If a Ti or Ri plasmid is to be used for the transformation, at least the right border, but in most cases the right and left border, of the Ti or Ri plasmid T-DNA is linked to the expression cassette to be introduced as flanking region.

Binary vectors are preferably used. Binary vectors are capable of replication both in E. coli and in Agrobacterium. As a rule, they comprise a selection marker gene and a linker or polylinker flanked by the right and left T-DNA border sequence. They can be transformed directly into Agrobacterium (Holsters et al., (1978) Mol Gen Genet. 163:181-187). The selection marker gene, which is, for example, the nptII gene, which confers resistance to kanamycin, permits a selection of transformed agrobacteria. The agrobacterium which acts as host organism in this case should already comprise a plasmid with the vir region. The latter is required for transferring the T-DNA to the plant cells. An agrobacterium transformed in this way can be used for transforming plant cells. The use of T-DNA for the transformation of plant cells has been studied intensively and described (EP 120 516; Hoekema, In: The Binary Plant Vector System, Offsetdrukkerij Kanters B. V., Alblasserdam, Chapter V; An et al. (1985) EMBO J. 4:277-287). Various binary vectors, some of which are commercially available, such as, for example, pBI1.2 or pBIN19 (Clontech Laboratories, Inc. USA), are known.

Further promoters which are suitable for expression in plants have been described (Rogers et al. (1987) Meth in Enzymol 153:253-277; Schardl et al. (1987) Gene 61:1-11; Berger et al. (1989) Proc Natl Acad Sci USA 86:8402-8406).

Direct transformation techniques are suitable for any organism and cell type. In cases where DNA or RNA are injected or electroporated into plant cells, the plasmid used need not meet any particular requirements. Simple plasmids such as those from the pUC series may be used. If intact plants are to be regenerated from the transformed cells, it is necessary for an additional selectable marker gene to be present on the plasmid.

Stably transformed cells, i.e. those which comprise the inserted DNA integrated into the DNA of the host cell, can be selected from untransformed cells when a selectable marker is part of the inserted DNA. By way of example, any gene which is capable of conferring resistance to antibiotics or herbicides (such as kanamycin, G 418, bleomycin, hygromycin or phosphinothricin and the like) is capable of acting as marker (see above). Transformed cells which express such a marker gene are capable of surviving in the presence of concentrations of such an antibiotic or herbicide which kill an untransformed wild type. Examples are mentioned above and preferably comprise the bar gene, which confers resistance to the herbicide phosphinothricin (Rathore K S et al. (1993) Plant Mol Biol 21(5):871-884), the nptII gene, which confers resistance to kanamycin, the hpt gene, which confers resistance to hygromycin, or the EPSP gene, which confers resistance to the herbicide glyphosate. The selection marker permits selection of transformed cells from untransformed cells (McCormick et al, (1986) Plant Cell Reports 5:81-84). The plants obtained can be bred and hybridized in the customary manner. Two or more generations should be grown in order to ensure that the genomic integration is stable and hereditary.

The above-described methods are described, for example, in Jenes B et al. (1993) Techniques for Gene Transfer, in: Transgenic Plants, Vol. 1, Engineering and Utilization, edited by SD Kung and R Wu, Academic Press, pp. 128-143, and in Potrykus (1991) Annu Rev Plant Physiol Plant Molec Biol 42:205-225). The construct to be expressed is preferably cloned into a vector which is suitable for transforming Agrobacterium tumefaciens, for example pBin19 (Bevan et al. (1984) Nucl Acids Res 12:8711f).

Once a transformed plant cell has been generated, an intact plant can be obtained using methods known to the skilled worker. For example, callus cultures are used as the starting material. The development of shoot and root can be induced from this as yet undifferentiated cell biomass in the known fashion. The plantlets obtained can be planted out and used for breeding.

The skilled worker is familiar with such methods for regenerating plant parts and intact plants from plant cells. Methods which can be used for this purpose are, for example, those described by Fennell et al. (1992) Plant Cell Rep. 11: 567-570; Stoeger et al (1995) Plant Cell Rep. 14:273-278; Jahne et al. (1994) Theor Appl Genet. 89:525-533.

“Transgenic” or “recombinant” for example in the case of a nucleic acid sequence, an expression cassette or a vector comprising said nucleic acid sequence or an organism transformed with said nucleic acid sequence, expression cassette or vector, refers to all those constructs established by recombinant methods in which either
a) the nucleic acid sequence encoding a yeast G3PDH or
b) a genetic control sequence, for example a promoter which is functional in plant organisms, which is linked operably with said nucleic acid sequence under a), or
c) (a) and (b)
   are not in their natural genetic environment or have been modified by recombinant methods, it being possible for the modification to be, for example, a substitutions, additions, deletions, inversion or insertions of one or more nucleotide residues. Natural genetic environment refers to the natural chromosomal locus in the source organism or the presence in a genomic library. In the case of a genomic library, the natural genetic environment of the nucleic acid sequence is preferably retained, at least to some extent. The environment flanks the nucleic acid sequence at least on one side and has a sequence length of at least 50 bp, preferably at least 500 bp, especially preferably at least 1000 bp, very especially preferably at least 5000 bp. A naturally occurring expression cassette, for example the naturally occurring combination of the promoter of a gene encoding for a yeast G3PDH, becomes a transgenic expression cassette when the latter is modified by non-natural, synthetic (“artificial”) methods such as, for example, a mutagenic treatment. Such methods are described (U.S. Pat. No. 5,565,350; WO 00/15815; see also above).

Host or starting organisms which are preferred as transgenic organisms are, above all, plants in accordance with the above definition. Included for the purposes of the invention are all genera and species of monocotyledonous and dicotyledonous plants of the Plant Kingdom, in particular plants which are used for obtaining oils, such as, for example, oilseed rape, sunflower, sesame, safflower, olive tree, soya, maize and nut species. Furthermore included are the mature plants, seed, shoots and seedlings, and parts, propagation material and cultures, for example cell cultures, derived therefrom Mature plants refers to plants at any desired developmental stage beyond the seedling stage. Seedling refers to a young, immature plant at an early developmental stage.

The transgenic plants can be generated with the above-described methods for the transformation or transfection of organisms.

The invention furthermore relates to the direct use of the transgenic plants according to the invention and to the cells, cell cultures, parts—such as, for example, in the case of transgenic plants, roots, leaves and the like—and transgenic propagation material such as seeds or fruits which are derived therefrom for the production of foodstuffs or feedstuffs, cosmetics or pharmaceuticals, in particular oils, fats, fatty acids or derivatives of these. To this end, the plants or plant parts are added in the usual amounts to the foodstuffs, feedstuffs, cosmetics, pharmaceuticals or products with industrial applications. Also, it is possible to obtain the oils and/or if appropriate free fatty acids from the plants, preferably from the seeds, and to add them in the usual amounts to the foodstuffs, feedstuffs, cosmetics, pharmaceuticals or products with industrial applications.

Besides influencing the oil content, the transgenic expression of a yeast G3PDH in plants may impart yet further advantageous effects such as, for example, an increased stress resistance to, for example, osmotic stress. Via increased glycerol levels, the yeast G3PDH confers protection against this type of stress, with glycerol acting as osmoprotective substance. Such osmotic stress occurs for example in saline soils and water and is an increasing problem in agriculture. Increased stress tolerance makes it possible, for example, to use areas in which conventional arable plants are not capable of thriving for agricultural usage.

Furthermore, recombinant expression of the yeast G3PDH can influence the NADH level and thus the redox balance in the plant organism. Stress such as, for example, drought, high or low temperatures, UV light and the like can lead to increased NADH levels and to an increased formation of reactive oxygen (RO). Transgenic expression of the yeast G3PDH can break down excessive NADH, which accumulates under said stress conditions, and thus stabilize the redox balance and alleviate the effects of the stress.

### Sequences

1. SEQ ID NO: 1
   Nucleic acid sequence coding for Saccharomyces cerevisiae G3PDH (Gpd1p)
2. SEQ ID NO: 2
   Protein sequence of the Saccharomyces cerevisiae G3PDH (Gpd1p)
3. SEQ ID NO: 3
   Nucleic acid sequence coding for Saccharomyces cerevisiae G3PDH (Gpd2p)
4. SEQ ID NO:4
   Protein sequence of the Saccharomyces cerevisiae G3PDH (Gpd2p)
5. SEQ ID NO: 5
   Protein sequence of the Saccharomyces cerevisiae G3PDH (Gpd2p) with second, alternative start codon
6. SEQ ID NO: 6
   Nucleic acid sequence coding for Schizosaccharomyces porn be G3PDH
7. SEQ ID NO: 7
   Protein sequence of the Schizosaccharomyces pombe G3PDH
8. SEQ ID NO: 8
   Nucleic acid sequence coding for Schizosaccharomyces pombe G3PDH
9. SEQ ID NO: 9
   Protein sequence of the Schizosaccharomyces pombe G3PDH
10. SEQ ID NO: 10
   Nucleic acid sequence coding for Yarrowinia lipolytica G3PDH
11. SEQ ID NO: 11
   Protein sequence of the Yarrowinia lipolytica G3PDH
12. SEQ ID NO: 12
   Protein sequence of the Yarrowinia lipolytica G3PDH with second, alternative start codon
13. SEQ ID NO: 13
   Nucleic acid sequence coding for Zygosaccharomyces rouxii G3PDH
14. SEQ ID NO: 14
   Protein sequence of the Zygosaccharomyces rouxii G3PDH
15. SEQ ID NO: 15
   Nucleic acid sequence coding for Zygosaccharomyces rouxii G3PDH
16. SEQ ID NO: 16
   Protein sequence of the Zygosaccharomyces rouxii G3PDH
17. SEQ ID NO: 17
   Expression vector based on pSUN-USP for S. cerevisiae G3PDH (Gpd1p; 1017-2190 bp insert)
18. SEQ ID NO: 18 Oligonucleotide primer OPN1

5′-ACTAGTATGTCTGCTGCTGCTGATAG-3′

19. SEQ ID NO: 19 Oligonucleotide primer OPN2

5′-CTCGAGATCTTCATGTAGATCTAATT-3′

20. SEQ ID NO: 20 Oligonucleotide primer OPN3

5′-GCGGCCGCCATGTCTGCTGCTGCTGATAG-3′

21. SEQ ID NO: 21 Oligonucleotide primer OPN4

5′-GCGGCCGCATCTTCATCTAGATCTAATT-3′

22-35: SEQ ID NP 22-35: Sequence motifs for yeast G3PDHs; possible sequence variations are given (see above). The variations of an individual motif may occur in each case alone, but also in the different combinations with each other.
36. SEQ ID NO: 36
   Expression vector pGPTV-gpd1 based on pGPTV-napin for S. cerevisiae G3PDH (Gpd1p; gdp1 insert of 11962-13137 bp; nos terminator: 13154-13408: napin promoter: 10807-11951).
37. SEQ ID NO: 37
   Nucleic acid sequence coding for Emericella nidulans G3PDH
38. SEQ ID NO: 38
   Protein sequence of the Emericella nidulans G3PDH
39. SEQ ID NO: 39
   Nucleic acid sequence coding for Debaryomyces hansenii G3PDH (partial)
40. SEQ ID NO: 40
   Protein sequence of the Debaryomyces hansenii G3PDH (partial)

### FIGURES

FIG. 1: Northern blot. Detection of the transcription of the yeast GPD1 gene in maturing seeds of transgenic oil seed rape lines (8, 6, 9 and 3). By way of comparison, the same detection has been carried out with wildtype (WT) plants. The GPD1 transcript was detected in lines 8, 6 and 9. In line 3, the GPD1 gene was not expressed. This line was employed in further analyses as additional control.

FIG. 2: Amount of glycerol 3-phosphate in maturing seeds (40 DAF=days after flowering) of transgenic GPD1 oil seed rape lines 8, 6 and 9 (black bars). By way of comparison, the content in corresponding, untransformed wild-type plants (WT) and of the nonexpressing transgenic line 3 (lighter bars) has been determined. The error deviations indicated are the result of in each case 6 independent measurements of all seeds obtained.

FIG. 3: Activity of glycerol 3-phosphate dehydrogenase in maturing seeds (40 DAF) of transgenic GPD1 oil seed rape lines 8, 6 and 9 (black bars). By way of comparison, the content in corresponding, untransformed wild-type plants (WT) and of the nonexpressing transgenic line 3 (lighter bars) has been determined. The error deviations indicated are the result of in each case 6 independent measurements of all seeds obtained.

FIG. 4: Total amount of lipids in the seeds of transgenic GPD1p oil seed rape fines (black bars) relative to the seed biomass. By way of comparison, the content in corresponding, untransformed wild-type plants (WT) and of the nonexpressing transgenic line 3 (lighter bars) has been determined. All 3 transgenic and expressing plants show a significant increase in the total amount of lipids in mature seeds. The error deviations indicated are the result of in each case 5 independent measurements of all seeds obtained.

FIG. 5 shows a sequence comparison of G3PDH homologs from other yeasts.

### TABLES

TABLE 1
Fatty acid profile of the seed oils in the GPD1p oil seed rape
lines 8, 6 and 9 (in mol %). By way of comparison, the fatty acid
profile in the corresponding untransformed wild-type plants (WT)
and of the nonexpressing transgenic line 3 is given.

| Fatty acid | Line 8 | Line 6 | Line 9 | WT | Line 3 |
|---|---|---|---|---|---|
| 16:0 | 5.3 ± 0.1 | 5.3 ± 0.3 | 5.1 ± 0.1 | 5.0 ± 0.1 | 5.6 ± 0.3 |
| 16:3 | 0.5 ± 0.02 | 0.47 ± 0.1 | 0.6 ± 0.2 | 0.5 ± 0.08 | 1.6 ± 0.0 |
| 18:0 | 1.1 ± 0.04 | 1.4 ± 0.3 | 1.1 ± 0.02 | 1.2 ± 0.13 | 1.5 ± 0.15 |
| 18:1 | 56.9 ± 1.6 | 58.0 ± 7.0 | 61.1 ± 1.6 | 60.6 ± 2.2 | 56.6 ± 2.5 |
| 18:2 | 25.3 ± 1.4 | 27.8 ± 3.3 | 23.7 ± 1.0 | 24.4 ± 1.5 | 24.9 ± 1.5 |
| 18:3 | 9.4 ± 0.4 | 12.1 ± 4.5 | 7.1 ± 0.5 | 7.2 ± 0.7 | 9.5 ± 1.0 |
| 20:0 | 0.4 ± 0.01 | 0.45 ± 0.1 | 0.4 ± 0.01 | 0.3 ± 0.05 | 0.6 ± 0.05 |

### General Methods:

Unless otherwise specified, all chemicals were from Fluka (Buchs), Merck (Darmstadt), Roth (Karlsruhe), Serva (Heidelberg) and Sigma (Deisenhofen). Restriction enzymes, DNA-modifying enzymes and molecular biology kits were from Amersham-Pharmacia (Freiburg), Biometra (Göttingen), Roche (Mannheim), New England Biolabs (Schwalbach), Novagen (Madison, Wis., USA), Perkin-Elmer (Weiterstadt), Qiagen (Hilden), Stratagen (Amsterdam, Netherlands), Invitrogen (Karlsruhe) and Ambion (Cambridgeshire, United Kingdom). The reagents used were employed in accordance with the manufacturer's instructions.

For example, oligonucleotides can be synthesized chemically in the known manner using the phosphoamidite method (Voet, Voet, 2nd edition, Wiley Press New York, pages 896-897). The cloning steps carried out for the purposes of the present invention such as, for example, restriction cleavages, agarose gel electrophoresis, purification of DNA fragments, transfer of nucleic acids to nitrocellulose and nylon membranes, linking DNA fragments, transformation of E. coli cells, bacterial cultures, multiplication of phages and sequence analysis of recombinant DNA, are carried out as described by Sambrook et al. (1989) Cold Spring Harbor Laboratory Press; ISBN 0-87969-309-6. Recombinant DNA molecules are sequenced using an ABI laser fluorescence DNA sequencer following the method of Sanger (Sanger et al. (1977) Proc Natl Acad Sci USA 74:5463-5467).

### EXAMPLE 1

### Cloning the Yeast Gpd1 Gene

Genomic DNA from Saccharomyces cerevisiae S288C (Mat alpha SUC2 mal mel gal2 CUP1 flo1 flo8-1; Invitrogen, Karlsruhe, Germany) was isolated following the protocol described hereinbelow:

A 100 ml culture was grown at 30° C. to an optical density of 1.0. 60 ml of the culture were spun down for 3 minutes at 3000×g. The pellet was resuspended in 6 ml of twice-distilled H₂O and the suspension was divided between 1.5 ml containers and spun down, and the supernatant was discarded. The pellets were resuspended in 200 μl of solution A, 200 μl phenol/chloroform (1:1) and 0.3 g of glass beads by vortexing and then lysed. After addition of 200 μl of TE buffer, pH 8.0, the lysates were spun for 5 minutes. The supernatant was subjected to ethanol precipitation with 1 ml of ethanol. After the precipitation, the resulting pellet was dissolved in 400 μl of TE buffer pH 8.0+30 μg/ml RNaseA. Following incubation for 5 minutes at 37° C., 18 μl 3 M sodium acetate solution pH 4.8 and 1 ml of ethanol were added, and the precipitated DNA was pelleted by spinning. The DNA pellet was dissolved in 25 μl of twice-distilled H₂O. The concentration of the genomic DNA was determined by its absorption at 260 nm.

### Solution A:

### 2% Trition-X100

### 1% SDS

### 0.1 M NaCl

### 0.01 M Tris-HCl pH 8.0

### 0.001 M EDTA

To clone the Gpd1 gene, the yeast DNA which has been isolated was employed in a PCR reaction with the oligonucleotide primers ONP1 and ONP2.

Sequence primer pair 1:
5′-ACTAGTATGTCTGCTGCTGCTGATAG
Sequence primer pair 2:
5′-CTCGAGATCTTCATGTAGATCTAATT

### Composition of the PCR Reaction (50 μl):

5.00 μl 5 μg genomic yeast-DNA
5.00 μl 10× buffer (Advantage polymerase)+25 mM MgCl₂
5.00 μl 2 mM dNTP
1.25 μl each primer (10 pmol/uL)
0.50 μl Advantage polymerase

The Advantage polymerase employed was from Clontech.

### PCR-Program:

Initial denaturation for 2 min at 95° C., then 35 cycles of 45 sec at 95° C., 45 sec at 55° C. and 2 min at 72° C. Final extension for 5 min at 7200.

The PCR products were cloned into the vector pCR2.1-TOPO (Invitrogen) following the manufacturer's instructions, resulting in the vector pCR2.1.-gpd1, and the sequence was verified by sequencing.

Cloning into the agrotransformation vector pGPTV involved incubating 0.5 μg of the vector pCR2.1-gpd1 with the restriction enzyme XhoI (New England Biolabs) for 2 hours and subsequent incubation for 15 minutes with Klenow fragment (New England Biolabs). After incubation for 2 hours with SpeI, the DNA fragments were separated by gel electrophoresis. The 1185 bp segment of the gpd1 sequence next to the vector (3.9 kb) was cut out from the gel, purified with the “Gel Purification” kit from Qiagen following the manufacturer's instructions and eluted with 50 μl of elution buffer. 0.1 μg of the vector pGPTV was first digested for 1 hour with the restriction enzyme SacI and then incubated for 15 minutes with Klenow fragment (New England Biolabs). 10 μl of the eluate of the gpd1 fragment and 10 ng of the treated pGPTV vector were ligated overnight at 16° C. (T4 ligase, New England Biolabs). The ligation products are then transformed into TOP10 cells (Stratagene) following the manufacturer's instructions and suitably selected, resulting in the vector pGPTV-gpd1. Positive clones are verified by sequencing and PCR using the primers 1 and 2.

To generate the vector pSUN-USP-gpd1, a PCR was carried out with the vector pCR2.1-gpd1 using the primers 3 and 4.

Sequence primer 3:
5′-GCGGCCGCCATGTGTGCTGCTGCTGATAG
Sequence primer 4:
5′-GCGGCCGCATCTTCATGTAGATCTAATT

### Composition of the PCR Reaction (50 μl):

5 ng DNA plasmid pCR2.1-gpd1
5.00 μl 10× buffer (Advantage polymerase)+25 mM MgCl₂
500 μl 2 mM dNTP
1.25 μl each primer (10 pmol/μl)
0.50 μl Advantage polymerase

The Advantage polymerase employed was from Clontech.

### PCR-Program:

Initial denaturation for 2 min at 95° C., then 35 cycles of 45 sec at 95° C., 45 sec at 55° C. and 2 min at 72° C. Final extension for 5 min at 72° C.

The 1190 bp PCR product was digested for 24 hours with the restriction enzyme NotI. The vector pSUN-USP was digested for 2 hours with NotI and then incubated for 15 minutes with alkaline phosphatase (New England Biolabs). 100 ng of the pretreated gpd1 fragment and 10 ng of the treated vector pGPTV were ligated overnight at 16° C. (T4 ligase, New England Biolabs). The ligation products are then transformed into TOP10 cells (Stratagene) following the manufacturer's instructions and suitably selected, resulting in the vector pSUN-USP-gpd1, Positive clones are verified by sequencing and PCR using the primers 3 and 4.

### EXAMPLE 2

### Plasmids for the Transformation of Plants

Binary vectors such as pBinAR can be used for the transformation of plants (Höfgen and Willmitzer (1990) Plant Science 66: 221-230), The binary vectors can be constructed by ligating the cDNA into T-DNA in sense or antisense orientation. 5′ of the cDNA, a plant promoter activates the transcription of the cDNA. A polyadenylation sequence is located 3′ of the cDNA.

Tissue-specific expression can be achieved using a tissue-specific promoter. For example, seed-specific expression can be achieved by cloning the napin or the LeB4- or the USP promoter 5′ of the cDNA. Any other seed-specific promoter element can also be used. The CaMV ³⁵S promoter can be used for constitutive expression in the whole plant.

A further example of binary vectors is the vector pSUN-USP and pGPTV-napin, into which the fragments the fragment of Example 2 was cloned. The vector pSUN-USP comprises the USP promoter and the OCS terminator. The vector pGPTV-napin comprises a truncated version of the napin promoter, and the nos terminator.

The fragments of Example 2 were cloned into the multiple cloning site of the vector pSUN-USP and pGPTV-napin respectively, to make possible the seed-specific expression of GPD1 The corresponding construct pSUN-USP-gpd1 is described by the SEQ ID NO: 16, and the construct of G3PDH in pGPTV-napin by SEQ ID NO: 36.

### EXAMPLE 3

### Transformation of Agrobacterium

Agrobacterium-mediated plant transformation can be carried out for example using the Agrobacterium tumefaciens strains GV3101 (pMP90) (Koncz and Schell (1986) Mol Gen Genet. 204: 383-396) or LBA4404 (Clontech). Standard transformation techniques may be used for the transformation (Deblaere et al. (1984) Nucl Acids Res 13:4777-4788).

### EXAMPLE 4

### Transformation of Plants

Agrobacterium-mediated plant transformation was effected using standard transformation and regeneration techniques (Gelvin Stanton B., Schilperoort Robert A., Plant Molecular Biology Manual, 2nd ed., Dordrecht: Kluwer Academic Publ., 1995, in Sect., Ringbuch Zentrale Signatur: BT11-P ISBN 0-7923-2731-4; Glick Bernard R., Thompson John E., Methods in Plant Molecular Biology and Biotechnology, Boca Raton: CRC Press, 1993, 360 pp., ISBN 0-8493-5164-2).

For example, oilseed rape was transformed by cotyledon or hypocotyl transformation (Moloney et al. (1989) Plant Cell Report 8:238-242; De Block et al. (1989) Plant Physiol 91: 694-701). The use of antibiotics for the selection of agrobacteria and plants depends on the binary vector used for the transformation and the agrobacterial strain. The selection of oilseed rape was carried out using kanamycin as selectable plant marker.

Agrobacterium-mediated gene transfer into linseed (Linum usitatissimum) can be carried out for example using a technique described by Mlynarova et al. (1994) Plant Cell Report 13:282-285.

Soya can be transformed for example using a technique described in EP-A-0 0424 047 (Pioneer Hi-Bred International) or in EP-A-0 0397 687, U.S. Pat. No. 5,376,543, U.S. Pat. No. 5,169,770 (University of Toledo).

The transformation of plants using particle bombardment, polyethylene glycol-mediated DNA uptake or via the silicon carbonate fiber technique is described, for example, by Freeling and Walbot “The Maize Handbook” (1993) ISBN 3-540-97826-7, Springer Verlag New York).

### EXAMPLE 5

### Studying the Expression of a Recombinant Gene Product in a Transformed Organism

A suitable method for determining the level of transcription of the gene (which indicates the amount of RNA available for translating the gene product) is to carry out a Northern blot as described hereinbelow (for reference see Ausubel et al. (1988) Current

Protocols in Molecular Biology, Wiley: New York, or the above examples section), where a primer which is designed such that it binds to the gene of interest is labeled with a detectable label (usually a radiolabel or a chemiluminescent label) so that, when the total RNA of a culture of the organism is extracted, separated on a gel, transferred to a stable matrix and incubated with this probe, the binding and the extent of the binding of the probe indicates the presence and also the amount of mRNA for this gene. This information indicates the degree of transcription of the transformed gene. Cellular total RNA can be prepared from cells, tissues or organs using several methods, all of which are known in the art, for example the method described by Bormann, E. R., et al, (1992) Mol. Microbiol. 6:317-326.

### Northern Hybridization:

To carry out the RNA hybridization, 20 μg of total RNA or 1 μg of poly(A)⁺ RNA were separated by means of gel electrophoresis in 1.25% strength agarose gels using formaldehyde and following the method described by Amasino (1986, Anal. Biochem. 152, 304), transferred to positively charged nylon membranes (Hybond N+, Amersham, Brunswick) by capillary force using 10×SSC, immobilized by UV light and prehybridized for 3 hours at 68° C. using hybridization buffer (10% dextran sulfate w/v, 1 M NaCl, 1% SDS, 100 mg herring sperm DNA). The DNA probe was labeled with the Highprime DNA labeling kit (Roche, Mannheim, Germany) during the prehybridization step, using alpha-³²P-dCTP (Amersham Pharmacia, Brunswick, Germany). Hybridization was carried out overnight at 68° C. after addition of the labeled DNA probe in the sa me buffer. The wash steps were carried out twice for 15 minutes using 2×SSC and twice for 30 minutes using 1×SSC, 1% SDS, at 68° C. The sealed filters were exposed at −70° C. for a period of 1 to 14 days.

To study the presence or the relative amount of protein translated from this mRNA, standard techniques such as a Western blot may be employed (see, for example, Ausubel et al. (1988) Current Protocols in Molecular Biology, Wiley: New York). In this method, the cellular total proteins are extracted, separated by means of gel electrophoresis, transferred to a matrix like nitrocellulose and incubated with a probe such as an antibody which binds specifically to the desired protein. This probe is usually provided with a chemiluminescent or calorimetric label which can be detected readily. The presence and the amount of the label observed indicates the presence and the amount of the desired mutated protein which is present in the cell.

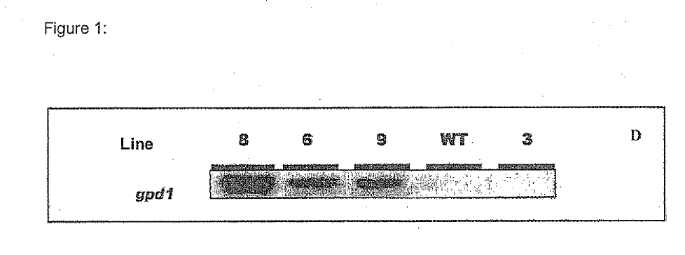

FIG. 1 shows the results of the Northern Blot of 4 independent transgenic oilseed rape lines and of the wild type. The plants of lines 6, 8 and 9 show a pronounced detection signal in the Northern Blot. Accordingly, the plants express the GPD1 gene in maturing seeds. In contrast, no transcription of the GPD1 gene was detected in the seed sample of line 3, which, in addition to the wild type, served as additional control, Moreover, line 3 demonstrates that the expression of the transferred gene is not successful in every single case, depending on the integration site in the genome of Brassica napus.

### EXAMPLE 6

### Analysis of the Effect of the Recombinant Proteins on the Production of the Desired Product

The effect of genetic modification in plants or on the production of a desired compound (such as a fatty acid) can be determined by growing the modified plant under suitable conditions (such as those described above) and examining the medium and/or the cellular components for increased production of the desired product (i.e. lipids or a fatty acid). These analytical techniques are known to the skilled worker and comprise spectroscopy, thin-layer chromatography, various staining methods, enzymatic and microbiological methods, and analytical chromatography such as high-performance liquid chromatography (see, for example, Ullmann, Encyclopedia of Industrial Chemistry, vol. A2, pp. 89-90 and pp. 443-613, VCH: Weinheim (1985); Fallon A et al. (1987) “Applications of HPLC in Biochemistry” in: Laboratory Techniques in Biochemistry and Molecular Biology, vol. 17; Rehm et al. (1993) Biotechnology, vol. 3, chapter III: “Product recovery and purification”, pp. 469-714, VCH: Weinheim; Belter P A et al. (1988) Bioseparations: downstream processing for Biotechnology, John Wiley and Sons; Kennedy J F and Cabral J M S (1992) Recovery processes for biological Materials, John Wiley and Sons; Shaeiwitz J A and Henry J D (1988) Biochemical Separations, in: Ullmann's Encyclopedia of Industrial Chemistry, vol. B3; chapter 11, p. 1-27, VCH: Weinheim, and Dechow, F. J. (1989) Separation and purification techniques in biotechnology, Noyes Publications).

In addition to the abovementioned methods, plant lipids are extracted from plant material as described by Cahoon et al. (1999) Proc. Natl. Acad. Sci. USA 96 (22):12935-12940, and Browse et al. (1986) Analytic Biochemistry 152:141-145. Qualitative and quantitative lipid or fatty acid analysis is described by Christie, William W., Advances in Lipid Methodology, Ayr/Scotland: Oily Press (Oily Press Lipid Library; 2); Christie, William W., Gas Chromatography and Lipids. A Practical Guide—Ayr, Scotland: Oily Press, 1989, Repr. 1992, IX, 307 pp. (Oily Press Lipid Library; 1); “Progress in Lipid Research, Oxford: Pergamon Press, 1 (1952)-16 (1977) under the title: Progress in the Chemistry of Fats and Other Lipids CODEN.

One example is the analysis of fatty acids (abbreviations: FAME, fatty acid methyl esters; GC-MS, gas-liquid chromatography/mass spectrometry; TAG, triacyiglycerol; TLC, thin-layer chromatography).

Unambiguous proof for the presence of fatty acid products can be obtained by analyzing recombinant organisms by analytical standard methods: GC, GC-MS or TLC, as described variously by Christie and the references cited therein (1997, in: Advances on Lipid Methodology, fourth edition: Christie, Oily Press, Dundee, 119-169; 1998, Gaschromatographie-Massenspektrometrie-Verfahren [gas-chromatographic/mass-spectrometric methods], Lipide 33:343-353).

The material to be analyzed can be disrupted by sonication, milling in the glass mill, liquid nitrogen and milling or other applicable methods. After disruption, the material must be centrifuged. The sediment is resuspended in distilled water, heated for 10 minutes at 100° C., cooled on ice and recentrifuged, followed by extraction in 0.5 M sulfuric acid in methanol with 2% dimethoxypropane for 1 hour at 90° C., which gives hydrolyzed oil and lipid compounds, which give transmethylated lipids. These fatty acid methyl esters are extracted in petroleum ether and finally subjected to GC analysis using a capillary column (Chrompack, WCOT Fused Silica, CP-Wax-52 CB, 25 μm, 0.32 mm) at a temperature gradient of between 170° C. and 240° C. for 20 minutes and for 5 minutes at 240° C. The identity of the fatty acid methyl esters obtained must be defined using standards which are available from commercial sources (i.e. Sigma).

Plant material is first homogenized mechanically by comminuting in a mortar to make it more accessible to extraction.

The following protocol was used for the quantitative oil analysis of the Brassica plants transformed with the Gpd1 gene:

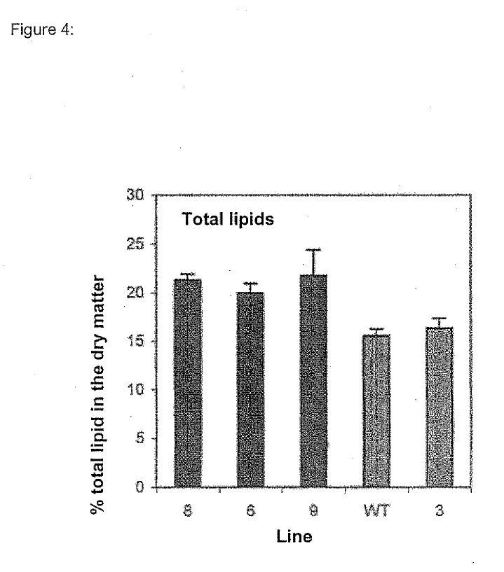

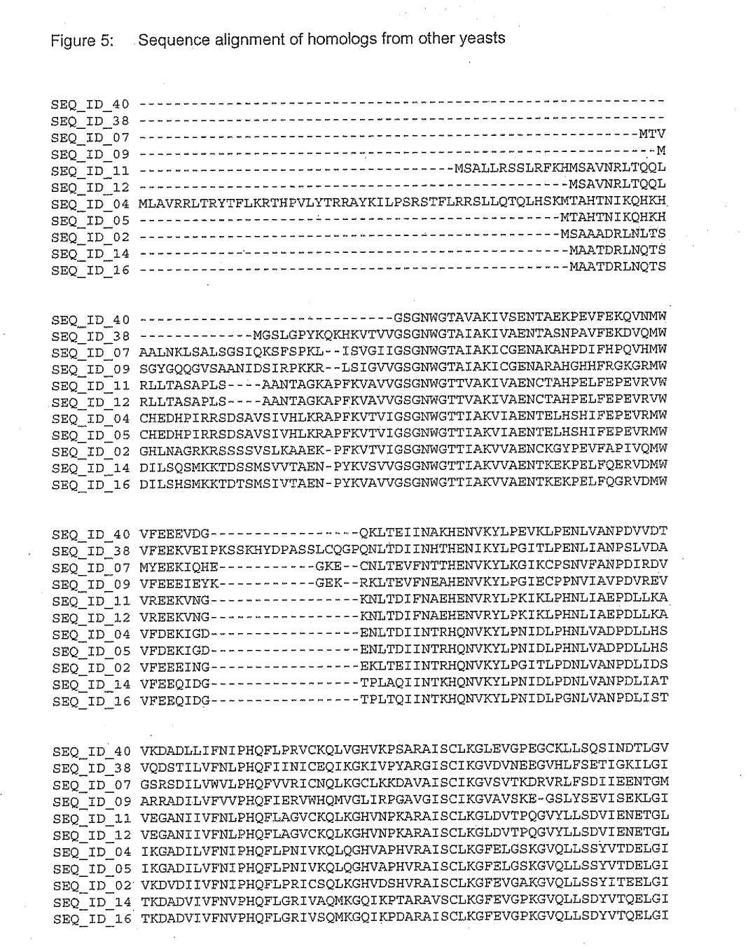

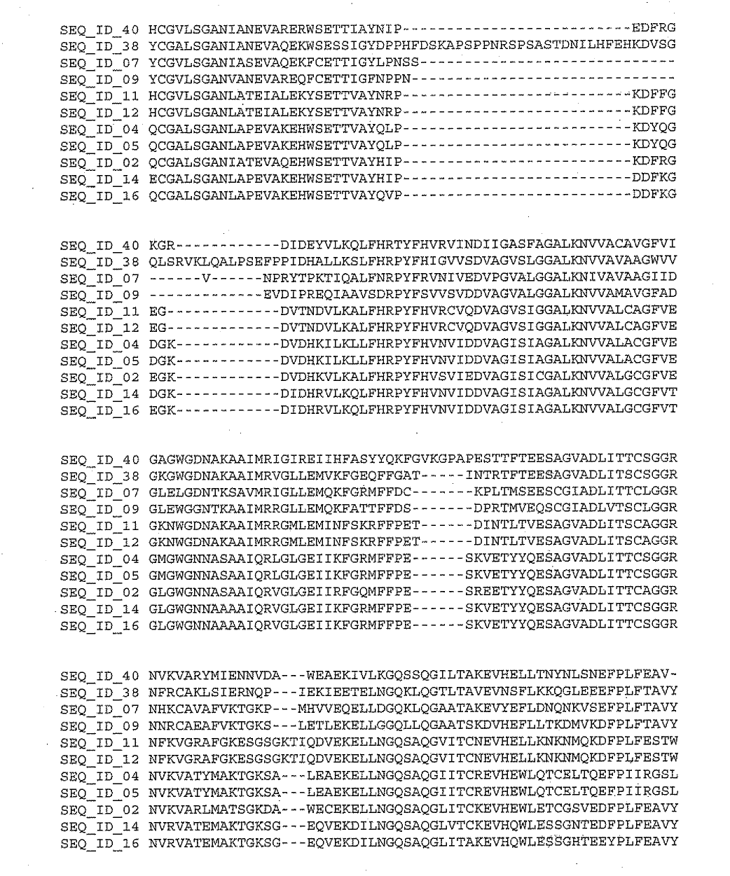

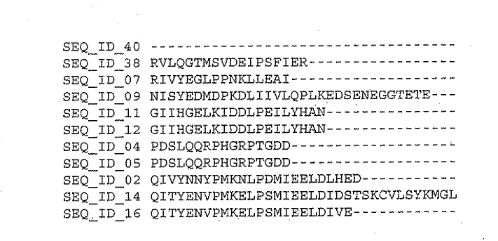

Lipid extraction from the seeds is carried out by the method of Bligh & Dyer (1959) Can J Biochem Physiol 37:911. To this end, 5 mg of Arabidopsis Brassica seeds are weighed into 1.2 ml Qiagen microtubes (Qiagen, Hilden) Using a Sartorius (Göttingen) microbalance. The seed material is homogenized with 1 ml chloroform/methanol (1:1; contains mono-C17-glycerol from Sigma as internal standard) in an MM300 Retsch mill from Retsch (Haan) and incubated for 20 minutes at RT. After centrifugation, the supernatant was transferred into a fresh vessel, and the sediment was re-extracted with 1 ml of chloroform/methanol (1:1). The supernatants were combined and evaporated to dryness. The fatty acids were derivatized by acidic methanolysis. To this end, the lipids which had been extracted were treated with 0.5 M sulfuric acid in methanol and 2% (v/v) dimethoxypropane and incubated for 60 minutes at 80° C. This was followed by two extractions with petroleum ether followed by wash steps with 100 mM sodium hydrogen carbonate and water. The fatty acid methyl esters thus prepared were evaporated to dryness and taken up in a defined volume of petroleum ether. 2 μl of the fatty acid methyl ester solution were finally separated by gas chromatography (HP 6890, Agilent Technologies) on a capillary column (Chrompack, WCOT Fused Silica, CP-Wax-52 CB, 25 m, 0.32 mm) and analysed via a flame ionization detector. The oil was quantified by comparing the signal intensities of the derivatized fatty acids with those of the internal standard mono-C17-glycerol (Sigma). By way of example, FIG. 4 shows the results for the quantitative determination of the oil contents in T3 seeds of 3 independent transgenic oilseed rape lines and of a nonexpressing control line and of the untransformed wild-type plants. Five independent extractions were carried out with the seed pools of each line, and the extracts were measured independently. The mean and the standard deviation were calculated from three independent measurements.

A significant increase in the total lipid content over that of the wild type and the nonexpressing control line was detected in the seed samples of transgenic lines 6, 8, and 9. This increase was between approximately 20 and 22% of the seed weight. In contrast, the lipid content in the wild type and in control line 3 were only approximately 15%. This corresponds to an increase in the total oil content by 33% or approximately 47%, respectively. In contrast, the fatty acid composition was not modified as a result of the GPD1 expression (see Table 1).

Table 1 shows the fatty acid composition in the T3 seeds of the transgenic GDH-expressing lines 8, 6 and 9 and of a nonexpressing control line 3 and of the untransformed wild-type plants. Five independent extractions were carried out with the seed pools of each line, and the extracts were measured independently. The mean and the standard deviation were calculated from the three independent measurements.

Oleic acid (18:1) accounts for the majority in the oil, with more than 55%, not only in the transgenic and expressing GDH lines 8, 6 and 9, but also in the nonexpressing control line 3 and in the untransformed wild-type plants.

### EXAMPLE 7

### Extraction of Glycerol 3-Phosphate

To extract glycerol 3-phosphate from maturing oilseed rape seeds, the latter are homogenized in an oscillatory mill (Retsch), treated with 500 μl of cold 16% (w/v) TCA/diethyl ether and incubated on ice for 20 minutes. Thereafter, 800 41 of cold 16% TCA/H2O, 5 mM EGTA are added and the mixture is incubated on ice for 3 hours. Insoluble components are sedimented by centrifugation. The liquid top phases are transferred to a fresh vessel, washed with 500 μl of cold, water-saturated diethyl ether at 4° C., and recentrifuged. The hydrophilic bottom phase is subjected to 3 more wash steps, and the pH is brought to 6-7 using 5 M KOH/1 M TEA. The hydrophilic phase is shock-frozen in liquid nitrogen, dried in a lyophilizer (Christ) and subsequently dissolved in 800 μl of H2O.

### EXAMPLE 8

### Determining the Amount of Glycerol 3-Phosphate (G3P)

The amount of G3P was determined by means of the enzymic cycling assay (Gibon et al. 2002). To this end, 10 μl of the hydrophilic phase (see above) or of the G3PDH replicates (see hereinbelow) are treated with 46 μl of Tricine/KOH (200 mM, pH 7.8)/10 mM MgCl2 and for 20 minutes at 95° C. in order to destroy the dihydroxyacetone phosphate. Thereafter, the samples are briefly subjected to incipient centrifugation, and the supernatant is treated with 45 μl of the reaction mixture (2 U glycerol 3-phosphate oxidase, 0.4 U glycerol 3-phosphate dehydrogenase, 130 U catalase, 0.12 μmol NADH). The reaction leads to a net consumption of NADH, which can be monitored directly on the photometer by the decrease of the absorption at 340 nm. The amount of G3P is calculated via a calibration line of different G3P concentrations.

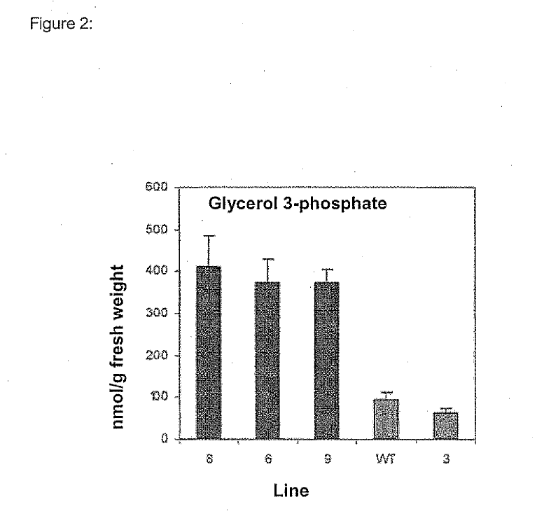

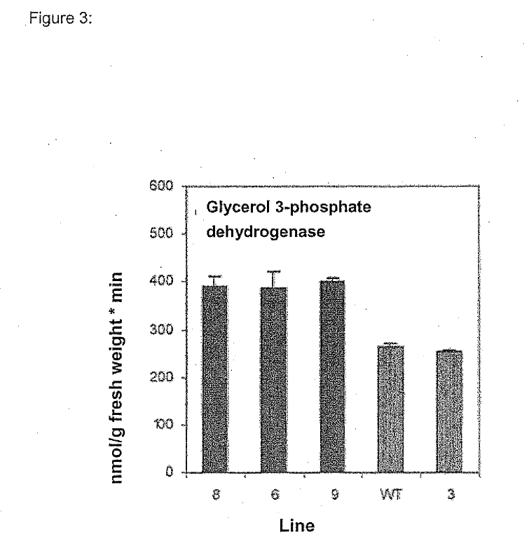

Interestingly, the seed-specific overexpression of GDP from Saccharomyces leads to a significant increase in the G3P content in maturing seeds of transgenic oilseed rape plants (40 DAF). The G3P contents in the seeds 40 DAF) of lines 6, 8 and 9 were between approximately 350 and 420 nmol G3P/g fresh weight. The G3P content in the wild-type seeds and in the seeds of the nonexpressing line, in contrast, was only between approximately 50 and 100 nmol 33 μg fresh weight (see FIG. 2).

### EXAMPLE 9

### Determination of the G3PDH Activity

To determine the G3PDH activity in the maturing oilseed rape seeds, the maturing seeds are isolated from frozen pods, weighed on a microbalance and homogenized using an oscillatory mill (Retsch). Thereafter, the samples are refrozen in liquid nitrogen.

Five hundred microliters of a cold extraction buffer (50 mM HEPES pH 7.4, 5 mM MgCl2, 1 mM EDTA, 1 mM EGTA, 5 mM dithiothreitol, 10% (v/v) glycerol, 2 mM benzamidine, 2 mM caproic acid, 0.5 mM phenylmethylsulfonyl fluoride, 1g/l polyvinylpolypyrrolidone) are added to the homogenized samples, mixed thoroughly and incubated for 30 minutes at 4° C. in the dark with continuous shaking. Thereafter, the samples are centrifuged for 15 minutes at 14000 rpm and 4° C. (Eppendorf centrifuge). The supernatant, which comprises the soluble proteins, is transferred into a fresh Eppendorf vessel and can be employed directly for determining the G3PDH activity or else used at −80° C.

10 μl of the protein extracts are pipetted together with 90 μl of reaction mixture (4 mM dihydroxyacetone phosphate; 0.2 mM NADH in 50 mM HEPES pH 7.4) and incubated for 30 minutes at 24° C. The reaction is then terminated by heating for 20 minutes at 95° C.

3 replications of each sample are employed for determining the G3PDH activity, one sample being heated directly and acting as blank. The amount of the glycerol 3-phosphate (G3P) formed is subsequently performed by the method of Gibon et al. 2002 (see above).

The transgenic lines 6, 8 and 9 which have tested positively at the transcription level showed a significantly increased glycerol 3-phosphate dehydrogenase activity in the maturing seeds (40 DAF) in comparison with the wild type or with the nonexpressing control line 3. In lines 6, 8 and 9, an activity of up to approximately 400 nmol G3P/g fresh weight and minute was detected. In the wild type and in the nonexpressing control line 3, in contrast, the activity only amounted to approximately 250 nmol G3P/g fresh weight and minute. This demonstrates that GPD1 is expressed in lines 6, 8 and 9 not only at the RNA level, but also at the enzyme level.

### EQUIVALENTS

The skilled worker recognizes or can identify many equivalents of the specific embodiments according to the invention described herein by merely performing routine experiments. These equivalents are to be comprised by the patent claims.

1. A method of increasing the total oil content in a transgenic oil crop plant, wherein the transgenic oil crop plant comprises at least 20% by weight of oleic acid based on the total fatty acid content and which comprises the following method steps:
a) introducing into an oil crop plant, a nucleic acid sequence which codes for a glycerol 3-phosphate dehydrogenase from a yeast, and
b) expressing, in the oil crop plant, the glycerol 3-phosphate dehydrogenase encoded by the nucleic acid, and
c) selecting an oil crop plant in which the total oil content is increased by at least 25% by weight in the plant in comparison with a nontransgenic plant.

2. The method of claim 1, wherein the total oil content is increased by at least 45% by weight in the plant in comparison with a nontransgenic plant.

3. The method according to claim 1, wherein the nucleic acid sequence which codes for the glycerol 3-phosphate dehydrogenase is derived from a yeast which is selected from the group consisting of the genera Cryptococcus, Torulopsis, Pityrosporum, Brettanomyces, Candida, Kloeckera, Trigonopsis, Trichosporon, Rhodotorul, Sporobolomyces, Bullera, Saccharomyces, Debaromyces, Lipomyces, Hansenula, Endomycopsis, Pichia and Hanseniaspora.

4. The method of claim 1, wherein the nucleic acid sequence which codes for the glycerol 3-phosphate dehydrogenase is derived from a yeast which is selected from the group consisting of Saccharomyces cerevisiae, Pichia pastoris, Hansenula polymorpha, Schizosaccharomyces pombe, Kluyveromyces lactis, Zygosaccharomyces rouxii, Yarrowia lipolitica, Emericella nidulans, Debaryomyces hansenii and Torulaspora hansenii.

5. The method of claim 1, wherein the glycerol 3-phosphate dehydrogenase which is encoded by the nucleic acid sequence brings about the conversion of dihydroxyacetone phosphate to glycerol 3-phosphate utilizing NADH or NADPH as cosubstrate and having a peptide sequence comprising at least one sequence motif selected from the group of sequence motifs consisting of

| | | |
|---|---|---|
| i) | GSGNWGT(A/T)IAK | (SEQ ID NO: 22) |
| ii) | CG(V/A)LSGAN(L/I/V)AXE(V/I)A | (SEQ ID NO: 26) |
| iii) | (L/V)FXRPYFXV. | (SEQ ID NO: 27) |

6. The method of claim 1, wherein the glycerol 3-phosphate dehydrogenase which is encoded by the nucleic acid sequence brings about the conversion of dihydroxyacetone phosphate to glycerol 3-phosphate utilizing NADH or NADPH as cosubstrate and having a peptide sequence comprising at least one sequence motif selected from the group of sequence motifs consisting of

| | | |
|---|---|---|
| i) | GSGNWGTTIAKV(V/I)AEN | (SEQ ID NO: 29) |
| ii) | NT(K/R)HQNVKYLP | (SEQ ID NO: 30) |
| iii) | D(I/V)LVFN(I/V)PHQFL | (SEQ ID NO: 31) |
| iv) | RA(I/V)SCLKGFE | (SEQ ID NO: 32) |
| v) | CGALSGANLA(P/T)EVA | (SEQ ID NO: 33) |
| vi) | LFHRPYEHV | (SEQ ID NO: 34) |
| vii) | GLGEII(K/R)FG. | (SEQ ID NO: 35) |

7. The method of claim 5, wherein the glycerol 3-phosphate dehydrogenase encoded by the nucleic acid sequence additionally comprises at least one sequence motif selected from the group of sequence motifs consisting of

| | | |
|---|---|---|
| i) | H(E/Q)NVKYL | (SEQ ID NO: 23) |
| ii) | (D/N)(I/V)(L/I)V(F/W)(V/N) | (SEQ ID NO: 24) |
| | (L/I/V)PHQF(V/L/I) | |
| iii) | (A/G)(I/V)SC(L/I)KQ | (SEQ ID NO: 25) |
| iv) | G(L/M)(L/G)E(M/I)(I/Q)(R/K/N) | (SEQ ID NO: 28) |
| | F(G/S/A). | |

8. The method according of claim 1, wherein the nucleic acid sequence encoding the glycerol 3-phosphate dehydrogenase is selected from the group consisting of:
a) a nucleic acid sequence encoding a polypeptide with the sequence shown in SEQ ID NO: 2, 4, 5, 7, 9, 11, 12, 14, 16, 38 or 40, or
b) a functional equivalent of a) which encodes a polypeptide with at least 60% identity with the sequence shown in SEQ ID NO: 2.

9. The method of claim 1, wherein, to express the nucleic acid sequence according to claim 1 (a) and (b), this sequence is operably linked with a promoter or terminator.

10. The method of claim 1, wherein the total oil content in the seed of the oil crop plant is increased.

11. The method according to claim 10, wherein the seed of the oil crop plant is harvested after growing the plant and, optionally, the oil present in the seed is isolated.

12. The method of claim 1, wherein the oil crop plant is selected from the group of oil crop plants consisting of Anacardium occidentale, Arachis hypogaea, Borago officinalis, Brassica campestris, Brassica napus, Brassica rapa, Brassica juncea, Camelina saliva, Cannabis sativa, Curthamus tinctorius, Cocos nucifera, Crambe abyssinica, Cuphea ciliata, Elaeis guineensis, Glycine max, Gossypium hirsitum, Gossypium barbadense, Gossypium herbaceum, Helianthus annus, Linum usitatissimum, Oenothera biennis, Olea europaea, Ricinus communis, Zea mays, Juglans regia and Prunus dulcis.

13. The method according to claim 1, wherein not only the total oil content is increased, but also the glycerol 3-phosphate content is increased by at least 20% by weight in the transgenic oil crop plant.

14. The method according to claim 11, wherein fatty acids present in the oil are liberated.

15. The method according to claim 10, wherein the oil or fatty acids which have been liberated are added to polymers, foodstuffs, feedstuffs, cosmetics, pharmaceuticals or products with industrial applications or employed as lubricants.

16. The method according to claim 2, wherein the nucleic acid sequence which codes for the glycerol 3-phosphate dehydrogenase is derived from a yeast which is selected from the group consisting of the genera Cryptococcus, Torulopsis, Pityrosporum, Brettanomyces, Candida, Kloeckera, Trigonopsis, Trichosporon, Rhodotorul, Sporobolomyces, Bullera, Saccharomyces, Debaromyces, Lipomyces, Hansenula, Endomycopsis, Pichia and Hanseniaspora.

17. The method of claim 2, wherein the glycerol 3-phosphate dehydrogenase which is encoded by the nucleic acid sequence brings about the conversion of dihydroxyacetone phosphate to glycerol 3-phosphate utilizing NADH or NADPH as cosubstrate and having a peptide sequence comprising at least one sequence motif selected from the group of sequence motifs consisting of

| | | |
|---|---|---|
| i) | GSGNWGT(A/T)IAK | (SEQ ID NO: 22) |
| ii) | CG(V/A)LSGAN(L/I/V)AXE(V/I)A | (SEQ ID NO: 26) |
| iii) | (L/V)FXRPYFXV. | (SEQ ID NO: 27) |

18. The method of claim 2, wherein the glycerol 3-phosphate dehydrogenase which is encoded by the nucleic acid sequence brings about the conversion of dihydroxyacetone phosphate to glycerol 3-phosphate utilizing NADH or NADPH as cosubstrate and having a peptide sequence comprising at least one sequence motif selected from the group of sequence motifs consisting of

| | | |
|---|---|---|
| i) | GSGNWGTTIAKV(V/I)AEN | (SEQ ID NO: 29) |
| ii) | NT(K/R)HQNVKYLP | (SEQ ID NO: 30) |
| iii) | D(I/V)LVFN(I/V)PHQFL | (SEQ ID NO: 31) |
| iv) | RA(I/V)SCLKGFE | (SEQ ID NO: 32) |
| v) | CGALSGANLA(P/T)EVA | (SEQ ID NO: 33) |
| vi) | LFHRPYFHV | (SEQ ID NO: 34) |
| vii) | GLGEII(K/R)FG. | (SEQ ID NO: 35) |

19. The method of claim 6, wherein the glycerol 3-phosphate dehydrogenase encoded by the nucleic acid sequence additionally comprises at least one sequence motif selected from the group of sequence motifs consisting of

| | | |
|---|---|---|
| i) | H(E/Q)NVKYL | (SEQ ID NO: 23) |
| ii) | (D/N)(I/V)(L/I)V(F/W)(V/N) | (SEQ ID NO: 24) |
| | (L/I/V)PHQF(V/L/I) | |
| iii) | (A/G)(I/V)SC(L/I)KQ | (SEQ ID NO: 25) |
| iv) | G(L/M)(L/G)E(M/I)(I/Q)(R/K/N) | (SEQ ID NO: 28) |
| | F(G/S/A). | |

20. The method of claim 2, wherein the nucleic acid sequence encoding the glycerol 3-phosphate dehydrogenase is selected from the group consisting of:
a) a nucleic acid sequence encoding a polypeptide with the sequence shown in SEQ ID NO: 2, 4, 5, 7, 9, 11, 12, 14, 16, 38 or 40, or
b) a functional equivalent of a) which encodes a polypeptide with at least 60% identity with the sequence shown in SEQ ID NO: 2.
